(19) United States
(12) Patent Application Publication
(10) Pub. No.: US 2026/0308451 A1
(43) Pub. Date: Oct. 8, 2026

(54) ENGINEERED BIOLOGIC LIVING MATERIALS AND METHODS TO MAKE THESE VIA ADVANCED MANUFACTURING

(71) Applicant: Trustees of Tufts College, MEDFORD, MA (US)

(72) Inventors: David L. Kaplan, Concord, MA (US); Junqi Wu, Medford, MA (US); Zaira Martin Moldes, Newton, MA (US); Emily Jane Hartzell, Waltham, MA (US); Constancio Gonzalez-Obeso, Boston, MA (US); Yushu Wang, Medford, MA (US); Chunmei Li, Stoneham, MA (US)

(21) Appl. No.: 19/258,708

(22) Filed: Jul. 2, 2025

Related U.S. Application Data
(63) Continuation of application No. PCT/US24/10680, filed on Jan. 8, 2024.
(60) Provisional application No. 63/478,904, filed on Jan. 6, 2023.

(51) Int. Cl. C09K 17/32; C07K 14/435

(52) U.S. Cl. CPC C09K 17/32; C07K 14/43586

(57) ABSTRACT

The present disclosure provides engineered biologic living materials (EBLMs) and methods of making the same. EBLMs can include thermoplastically-molded silk that includes one or more microorganisms of interest that are biologically alive and active embedded within the silk. The EBLMs expand the universe of living materials and are useful in a variety of fields, from probiotic delivery to soil treatment. The compositions disclosed provide a surprising degree of protection to the microorganisms, allowing them to endure conditions that would otherwise be destructive to the microorganisms, including high temperatures and pressures experienced during thermoplastic molding.

### CLAIM TO PRIORITY

This application claims benefit of and is a continuation of International Patent Application No. PCT/US2024/010680 (Attorney Docket. No. 2095.0582), filed Jan. 8, 2024, and entitled “ENGINEERED BIOLOGIC LIVING MATERIALS AND METHODS TO MAKE THESE VIA ADVANCED MANUFACTURING,” International Pub. No. WO2024148355, which is hereby incorporated by reference in its entirety for all purposes.

International Patent Application No. PCT/US2024/010680 relates to, incorporates by reference for all purposes, and claims priority to U.S. Application Ser. No. 63/478,904, filed Jan. 6, 2023.

### STATEMENT REGARDING FEDERALLY FUNDED RESEARCH

This invention was made with government support under FA9550-20-1-0363 awarded by the United States Air Force. The government has certain rights in the invention.

### SEQUENCE LISTING

A Sequence Listing accompanies this application and is submitted as an XML file of the sequence listing named “T002640 WO-2095.0582 Sequence Listing” which is 2,856 bytes in size and was created on Jan. 4, 2024. The sequence listing is electronically submitted with the application and is incorporated herein by reference in its entirety.

### BACKGROUND

Integrating biological systems into articles can sometimes be facile (e.g., when processing conditions are entirely conducive to healthy function of the biological systems), but many of the processing conditions used in conventional manufacturing tend to be deleterious to biological function. One particular challenging manufacturing environment for maintaining biologically alive and active systems is thermoplastic molding, because thermoplastic molding conventionally uses high pressures and/or high temperatures.

A need exists for a wider range of materials that are capable of serving a conventional material function (e.g., providing strength, exhibiting flexibility, etc.) while simultaneously maintaining within the material alive and active biological systems. A further needs exists for such systems that can be integrated within existing manufacturing approaches.

### SUMMARY

The viability and function of living bacteria including probiotics Escherichia coli Nissle 1917 (EcN) and soil bacteria Rhizobium tropici CIAT 899 (CIAT. 899) can be maintained after incorporation into regenerated silk articles using thermoplastic molding. With bacteria surviving from thermoplastic molding when protected by silk, disclosed herein is a one-step process to create silk-based dense materials with living functions. Thermoplastic molding enables facile manufacturing, material machinability, is inexpensive, and vastly expands material features, functions, and novelty.

The present disclosure includes, among various aspects and features, a thermoplastically-molded silk fibroin article having embedded therein a plurality of microorganisms of interest that are biologically alive and active. The present disclosure also includes a silk living material composition comprising a thermoplastically-molded silk fibroin article having embedded therein a plurality of microorganisms of interest that are biologically alive and active. The present disclosure further includes a self-healing silk composition comprising a thermoplastically-molded silk fibroin article having embedded therein a plurality of microorganisms of interest that are biologically alive and active. The present disclosure also includes a method comprising providing a mixture of silk fibroin and a plurality of microorganisms of interest and applying at least one of an elevated temperature and an elevated pressure to the silk fibroin-microorganism mixture to generate a thermoplastically-molded silk fibroin article having embedded therein the plurality of microorganisms of interest that are biologically alive and active. The present disclosure additionally includes a method of generating a silk living material comprising providing a mixture of silk fibroin and a plurality of microorganisms of interest and applying at least one of an elevated temperature and an elevated pressure to the mixture of silk fibroin and a plurality of microorganisms to generate a silk living material having embedded therein the plurality of microorganisms of interest that are biologically alive and active or a thermoplastically-molded silk fibroin article having embedded therein the plurality of microorganisms of interest that are biologically alive and active. In addition, the present disclosure provides a method of generating self-healing silk comprising providing a mixture of silk fibroin and a plurality of microorganisms of interest and applying at least one of an elevated temperature and an elevated pressure to the mixture of silk fibroin and a plurality of microorganisms to generate self-healing silk.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTIONS OF THE DRAWINGS

FIG. 1. Viability of E. coli after thermoplastic molding in silk plate. A) illustration of making silk living materials using the method of thermoplastic molding. B) viability test for silk fibroin no thermoplastic molding, silk fibroin thermoplastic molding, silk fibroin with ampicillin resistant E. coli incorporated no thermoplastic molding, and silk fibroin with ampicillin resistance E. coli incorporated thermoplastic molding at 100° C., 625 MPa. C) Viability test of ampicillin resistant E. coli after thermoplastic molding 100° C., 625 MPa. D) Fluorescent imaging of supernatant after overnight inoculation of E. coli-GFP contained silk plate compared with E. coli wild type contained in the silk plate and no bacteria contained in the silk plate as controls.

FIG. 2. Fluorescence microscopy images at excitation wavelength of 532 nm of silk plates containing E. coli expressing monomeric red fluorescent protein (mRFP). A) Fluorescence image of initial silk plate containing RFP-E. coli. B) Fluorescence image of silk plate containing mRFP-E. coli after 16 hours of inoculation. C) Fluorescence image of silk plate containing mRFP-E. coli after 4 days of inoculation. D) Fluorescent image of silk plate containing mRFP-E. coli with lactose added on day 5, imaged at day 6. Scale bar: 200 μm. E) SEM images of silk plate surface after inoculation for 6 days. F. SEM images of silk plate cross-section after inoculation for 6 days. Scale bar: 10 μm.

FIG. 3. Self-healing of silk plates with cavities. A) Illustration of creating cavities for silk living materials to demonstrate self-healing. B) digital images showing healed cavities for thermoplastic molded silk plates containing G. xylinus and the silk plate without thermoplastic molding. C) SEM images showing the growth of cellulose fiber in the previous cavity. D) digital image showing thermoplastic silk plate with no G. xylinus encapsulated with the cavity unchanged.

FIG. 4. Viability test of E. coli encapsulated silk from thermoplastic molding comparing with other biopolymers. A) viability testing results for thermoplastic molded biopolymer plates with E. coli encapsulated from top to bottom: silk fibroin, alginate, gelatin, soy, chitosan, cellulose and zein. B) digital images of thermoplastic molded plates. C) digital images of the plates after inoculation overnight. Scale bars: 1 cm.

FIG. 5. Protection of probiotics passage through the gastrointestinal tract with silk living materials. A) Genetic circuit design of pDawn plasmid for white light-activated RFP expression in EcN. B) SEM images of silk/REcN powders (i), WS/REcN with various magnifications (ii), and single REcN embedded by silk (iii). Scale bars in i, ii, insert in ii, and iii are 500 nm, 5 μm, 1 μm and 500 nm, respectively. C) Photograph of WS/REcN and scheme illustration (d) showing REcN embedded in the silk protection shell with high crystallinity. Scale bars, 5 mm.

FIG. 6. Protection of probiotics passage through the gastrointestinal tract with silk living materials. A) The bacterial counts of REcN exposed to LB Kan, SGF, and SGF+SIF (first in SGF then in SIF), respectively (n=5). B) Bacterial counts of RECN released from untreated WS/RECN and SGF or SGF+SIF-treated WS/REcN, respectively (n=5). C) Growth curves of the REcN cultured in LB Kan, SFG+SIF-treated REcN, REcN released from WS/REcN incubated in LB Kan, and RECN released from SGF+SIF treated WS/REcN, respectively, were determined by recording the change in OD600 with time (n=3).

FIG. 7. Fluorescence microscopy images of REcN and WS/REcN cultured in LB Kan or treated by SGF+SIF, respectively. Green: SYTO-9, Red: RFP. Scale bar, 100 μm.

FIG. 8. (a) Schematic of silk bioplastic degradation by protease secretion of endogenous rhizobium CIAT. 899 performed in the soil microorganism community environment. (Scheme needed to be improved) SEM images of silk/CIAT. 899 powders with enlarged image at right (b) and WS/CIAT. 899 (c). Scale bars in b (left), b (right), and c are 10 μm, 500 nm, and lum, respectively.

FIG. 9. A) Schematic and the optical microscopy images of the WS/60° C. and WS/CIAT. 899, showing proteolysis during 90-day soil degradation with CIAT. 899 embedded in the high crystalline content silk bioplastic compared with the nonliving silk bioplastic prepared with the same fabrication methods. Scale bar is 1 mm. b) Comparison of the degradation profile of WS/60° C. with soil and protease XIV and WS/CIAT. 899 in soil conditions. The shaded areas represent the degradation distribution range of each group (n=5).

FIG. 10. Cross-sectional SEM images of WS/60° C. and WS/CIAT. 899 at days 0, 30, 60, and 90 during soil degradation, and corresponding digital photographs inserted with original diameters of 10 mm at day 0. Scale bars are 100 μm.

FIG. 11. A) Fluorescence microscopy images of WS/60° C. and WS/CIAT. 899 at day 90 during the process of soil degradation. Green: SYTO-9. Scale bar, 100 μm. B) SEM image, and magnified image of WS/CIAT. 899 at day 90 during the process of soil degradation. Scale bars are 1 μm (left image) and 500 nm (right image).

FIG. 12. FEA results suggest the stress and heat distribution changes along the thickness of the silk/microbe living material system with time during thermal molding. Data points are shown as mean±s.d. *P<0.05, **P<0.01, ***P<0.001.

### DETAILED DESCRIPTION

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; and (v) where ranges are provided, endpoints are included.

Throughout this disclosure, the acronym ‘TS’ refers to thermal molded silk and the acronym ‘WS’ refers to water-plasticized, thermal molded silk.

Approximately: as used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biocompatible: the term “biocompatible”, as used herein, refers to materials that do not cause significant harm to living tissue when placed in contact with such tissue, e.g., in vivo. In certain aspects, materials are “biocompatible” if they are not toxic to cells. In certain aspects, materials are “biocompatible” if their addition to cells in vitro results in less than or equal to 20% cell death, and/or their administration in vivo does not induce significant inflammation or other such adverse effects.

Biodegradable: as used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain aspects, components generated by breakdown of a biodegradable material are biocompatible and therefore do not induce significant inflammation and/or other adverse effects in vivo. In some aspects, biodegradable polymer materials break down into their component monomers. In some aspects, breakdown of biodegradable materials (including, for example, biodegradable polymer materials) involves hydrolysis of ester bonds. Alternatively or additionally, in some aspects, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid) (PLA), poly(glycolic acid) (PGA), poly(lactic-co-glycolic acid) (PLGA), and copolymers with PEG, polyanhydrides, poly(ortho) esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Compaction: as used herein, the term “compaction” refers to a process by which a material progressively loses its porosity due to the effects of loading.

Composition: as used herein, may be used to refer to a discrete physical entity that comprises one or more specified components. In general, unless otherwise specified, a composition may be of any form—e.g., gas, gel, liquid, solid, etc. In some aspects, “composition” may refer to a combination of two or more entities for use in a single embodiment or as part of the same article. It is not required in all embodiments that the combination of entities result in physical admixture, that is, combination as separate co-entities of each of the components of the composition is possible; however many practitioners in the field may find it advantageous to prepare a composition that is an admixture of two or more of the ingredients in a pharmaceutically acceptable carrier, diluent, or excipient, making it possible to administer the component ingredients of the combination at the same time.

Fusion: as used herein, the term “fusion” refers to a process of combining two or more distinct entities into a new whole.

Hydrophilic: as used herein, the term “hydrophilic” and/or “polar” refers to a tendency to mix with, or dissolve easily in, water.

Hydrophobic: as used herein, the term “hydrophobic” and/or “non-polar”, refers to a tendency to repel, not combine with, or an inability to dissolve easily in, water.

Improve, increase, or reduce: as used herein or grammatical equivalents thereof, indicate values that are relative to a baseline measurement, such as a measurement in a similar composition made according to previously known methods.

Macroparticle: as used herein, the term “macroparticle” refers to a particle having a diameter of at least 1 millimeter. In some aspects, macroparticles are micelles in that they comprise an enclosed compartment, separated from the bulk solution by a micellar membrane, typically comprised of amphiphilic entities which surround and enclose a space or compartment (e.g., to define a lumen). In some aspects, a micellar membrane is comprised of at least one polymer, such as for example a biocompatible and/or biodegradable polymer. In some aspects, a population of particles is considered a population of macroparticles if the mean diameter of the population is equal to or greater than 1 millimeter.

Microparticle: as used herein, the term “microparticle” refers to a particle having a diameter between 1 micrometer and 1 millimeter. In some aspects, microparticles are micelles in that they comprise an enclosed compartment, separated from the bulk solution by a micellar membrane, typically comprised of amphiphilic entities which surround and enclose a space or compartment (e.g., to define a lumen). In some aspects, a micellar membrane is comprised of at least one polymer, such as for example a biocompatible and/or biodegradable polymer. In some aspects, a population of particles is considered a population of microparticles if the mean diameter of the population is between 1 micrometer and 1 millimeter.

Nanoparticle: as used herein, the term “nanoparticle” refers to a particle having a diameter of less than 1000 nanometers (nm). In some aspects, a nanoparticle has a diameter of less than 300 nm, as defined by the National Science Foundation. In some aspects, a nanoparticle has a diameter of less than 100 nm as defined by the National Institutes of Health. In some aspects, nanoparticles are micelles in that they comprise an enclosed compartment, separated from the bulk solution by a micellar membrane, typically comprised of amphiphilic entities which surround and enclose a space or compartment (e.g., to define a lumen). In some aspects, a micellar membrane is comprised of at least one polymer, such as for example a biocompatible and/or biodegradable polymer. In some aspects, a population of particles is considered a population of nanoparticles if the mean diameter of the population is equal to or less than 1000 nm.

Physiological conditions: as used herein, has its art-understood meaning referencing conditions under which cells or organisms live and/or reproduce. In some aspects, the term refers to conditions of the external or internal milieu that may occur in nature for an organism or cell system. In some aspects, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some aspects, conditions in a laboratory are manipulated and/or maintained at physiologic conditions. In some aspects, physiological conditions are encountered in an organism.

Pure: as used herein, a material, additive, and/or entity is “pure” if it is substantially free of other components. For example, a preparation that contains more than about 90% of a particular agent or entity is typically considered to be a pure preparation. In some aspects, an agent or entity is at least 91%, at least 92%, at least 93%, at least 94%, at least 95%, at least 96%, at least 97%, at least 98%, or at least 99% pure.

Reference: as used herein describes a standard or control relative to which a comparison is performed. For example, in some aspects, a material, article, additive, entity or other sample, sequence or value of interest is compared with a reference or control material, article, additive, entity or other sample, sequence or value. In some aspects, a reference or control is tested and/or determined substantially simultaneously with the testing or determination of interest. In some aspects, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Solid form: as is known in the art, many chemical entities (in particular many organic molecules and/or many small molecules) can adopt a variety of different solid forms such as, for example, amorphous forms and/or crystalline forms (e.g., polymorphs, hydrates, solvates, etc.). In some aspects, such entities may be utilized as a single such form (e.g., as a pure preparation of a single polymorph). In some aspects, such entities may be utilized as a mixture of such forms.

Substantially: as used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### Engineered Biologic Living Materials (EBLMs)

The inventors have developed a method to generate silk-based living materials using various thermoplastic molding techniques with controlled temperature and pressure. The inventors have found that regenerated silk fibroin can, surprisingly, stabilize and maintain the bioactivity of various types of living microorganisms including bacteria, fungi, and yeast at high temperatures and pressures when processed with silk, such that these organisms are alive and active in modifying the material after the thermoplastic molding process which exposes them to temperatures and/or pressures that would otherwise kill them. We show that the microorganisms within the processed silk fibroin maintained viability and function after thermoplastic pressure up to 625 MPa through plasticizer-assisted thermal molding methods. These findings provide a foundation to generate silk-based living materials using a one-step thermoplastic molding process to form novel living interfaces/materials with functionalities for applications in biosensing, self-healing, self-cleaning, dynamic interfaces, and long-lasting drug delivery. In all cases, sustainability of the materials, including 100% degradation, is part of the process.

Biomaterials have revolutionized society, from medicine to consumer goods and environmental systems. Conventional biomaterials are often static, inert materials that do not interact with the host or the environment-they are generally passive in function (e.g., barrier, transport, mechanics, display) and nonliving. Newer biomaterial systems have provided additional functions, such as through entrainment or immobilization of bioactive components, from drugs and enzymes to antibodies and other reporter systems. Previously, it was demonstrated that enzymes and antibiotics could be entrained in thermal molded silk devices and retain function-such as degradability and antimicrobial features. The inventors disclose herein ‘living materials’ with functional biological features such as growth, stimuli-response, growth materials, and many related features as living materials systems. These new innovative biomaterials are biologically active, instructive materials that host and provide signals to surrounding cells, tissues and the environment by encapsulating living cells (native or bioengineered variants of these cells for additional functions) into the material system. These new responsive, self-renewable, and functional biomaterials are termed ‘Engineered Biologic Living Materials’ (EBLMs), and are composed of two key components or parts: (a) the living entity (e.g., cells (responsive function)), and (b) the polymeric matrix (scaffolding function)) that must be combined in a processing mode to enable stabilization yet function of (a). Many engineered living materials have been developed, where bacteria, fungi, algae, and animal cells can be incorporated into the materials when in a loose, hydrogel-like, film-like or nanofiber formats. However, there is no evidence of dense materials formed via thermoplastic processing (e.g., high temperature and pressure) where living cells survive and function in these newly formed plastic-like materials. Such materials will be more durable and long lasting in the body or in environmental conditions and exposures, yet still fully degradable over time. Here, these living materials can be used as biosensors, skin patches for wound healing, drug delivery systems, new functional materials, coatings, anti-fouling systems, and for tissue engineering, among many other applications. In general, the disclosed innovations provide much more durable and plastic-like material features by combining the protective effects of the silk protein (during high temperatures and pressures) with biological function in living organisms.

Silk is a good candidate for living materials because of its biocompatibility and excellent physical and mechanical properties with versatile processability, including thermoplastic molding features. In addition, silk is known for its tunable beta sheet crystal structures, which stabilize biologics like proteins. Furthermore, silk has been widely used as a conventional biomaterial and shown applicability as a material for use in biosensors, drug delivery systems, and as scaffolds for tissue engineering. The development of silk living materials will significantly innovate using silk in these areas.

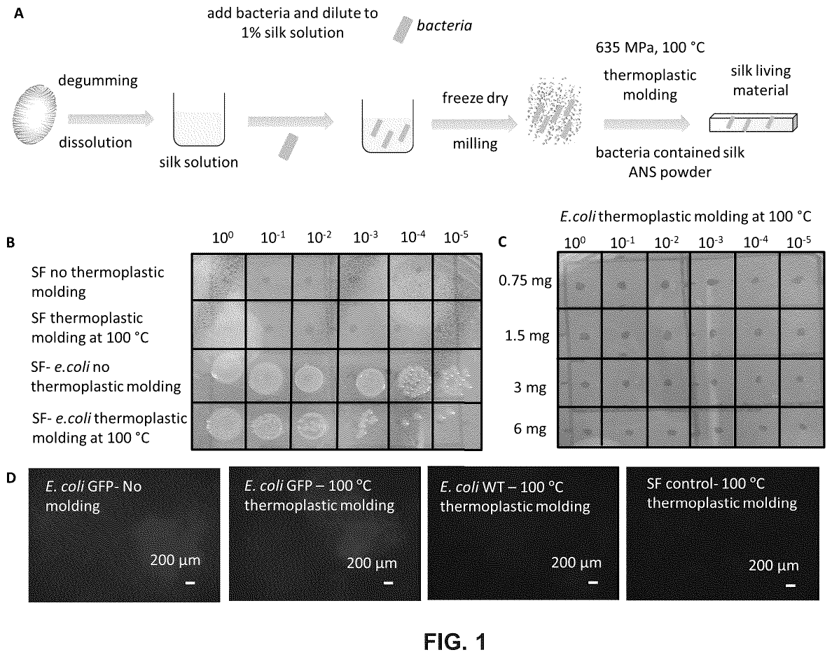

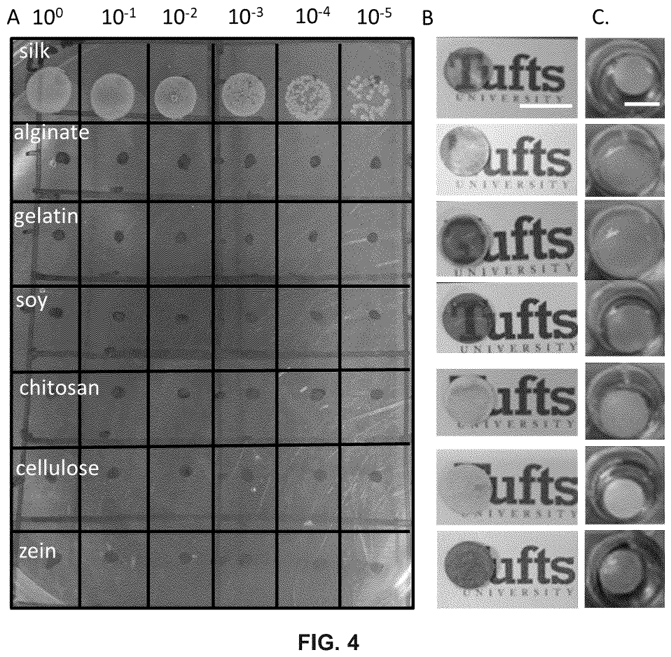

The inventors have found that microorganisms like bacteria, fungi, and yeast can be incorporated into silk fibroin materials by first dissolving the microorganism into silk aqueous solution, followed by freeze drying to generate precursor materials for processing. The viability of the microorganisms is then maintained after thermoplastic molding of the silk, and microorganisms mixed even up to 100° C. and 625 MPa remain biologically functional, see, for example, FIG. 1. If the same microorganisms are processed at these temperatures and pressures divorced from silk, they are killed and do not survive the processing, demonstrating the protective impact of silk for such entities. For example, the same microorganisms processed with cellulose, chitosan, gelatin, alginate, zein, and soy protein using the same thermoplastic molding conditions do not show viability after processing (FIG. 4).

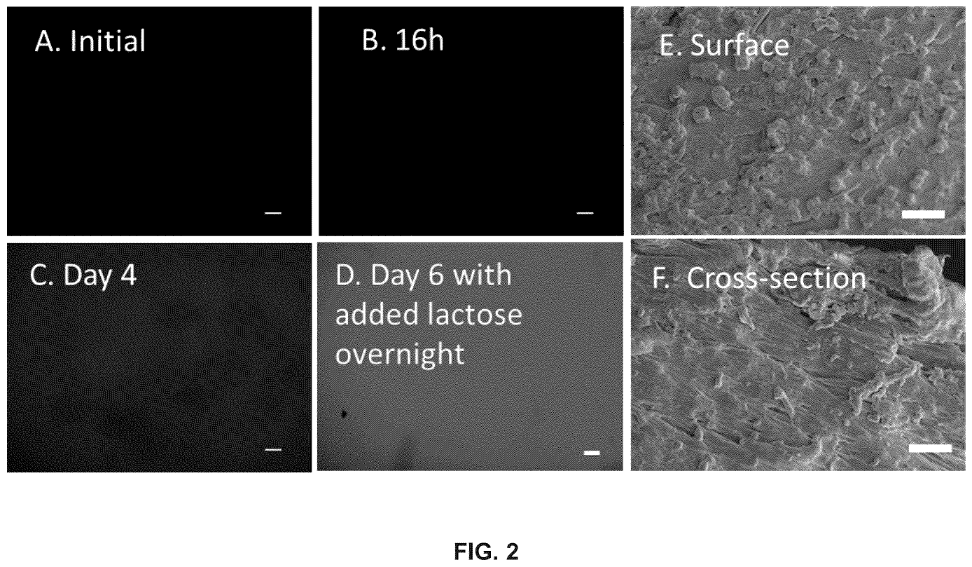

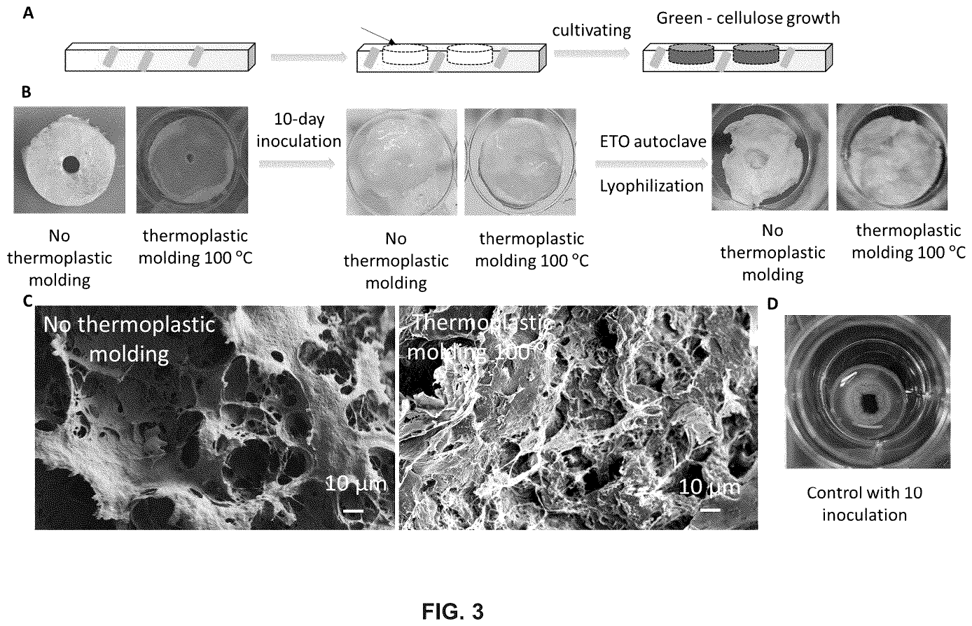

As an example, the inventors incorporated E. coli engineered to express red fluorescent protein (RFP) into silk fibroin using thermoprocessing to test if the entrapped bacteria maintained viability and function (expressing RFP). The inventors found that the red fluorescence of thermoplastic molded silk plates was enhanced after cultivation. When the thermoplastic molded (100° C. and 625 MPa) silk plates containing the bacteria were added to culture media containing lactose, a significant increase in fluorescent intensity was observed, as evidence that the E. coli's translational machinery was still functional after the thermoplastic molding (FIG. 2). As a second example, we incorporated the bacteria, Gluconacetobacter xylinus into silk fibroin using the same method as used above for E. coli. Before thermoplastic molding, we created a cavity in the middle of the precursor material, then thermoplastic molding was conducted to create the silk plate with cavity in the middle. After cultivation for 7 days, we found that the cavity has been healed by the growth of cellulose. A control of thermoplastic silk plates with a cavity, but without the bacterium, was place in the same media for the same time. No self-healing was observed without G. xylinus encapsulated in the silk material (FIG. 3). This experiment confirmed that the embedded bacteria was functional after thermoplastic molding at 100° C., 625 MPa.

In this disclosure, microorganisms were introduced into the thermoplastic process and the utility of these silk-based living material systems was assessed in two ways: probiotic delivery and protein-based material degradation. Probiotics can be used to balance the intestinal microbiome and compete with the growth of harmful bacteria, which enables the treatment of intestinal diseases and boosts the human immune system. However, probiotics, as living microorganisms usually face the inactivation issue in the harsh gastrointestinal environment with the presence of gastric acids and digestive enzymes. Thus, an efficient probiotic delivery system employing the new silk bioplastic living materials with high crystallinity was designed to serve as a protective shell to allow the sequestered microorganisms to resist environmental stressors and reach the large intestine. Furthermore, protease-secreting and nitrogen-fixing microorganisms were incorporated into the silk bioplastic as another living material paradigm for the soil environment, designed to degrade as an alternative to chemical fertilizers, realizing the sustainable recycling of protein-based materials.

Exploiting the power of biological systems (cells) to enhance material functions via traditional commodity plastics processing, such as thermoplastic molding, has eluded the materials technology community to date. The addition of such complex and utilitarian machines (cells) to harness their power, but within a materials contact, offers to transform functional materials of the future. The disclosed silk-based living materials could be useful for applications in biosensors, tissue engineering, self-healing materials, self-cleaning systems, and reporter functions, among others.

Many biopolymer based materials have been used to entrap biological cells in the past including agarose, alginate, and silk fibroin. Among these materials, many are processed into hydrogels for encapsulating cells with ambient processing conditions to stabilize the cells. However, the blocking of the nozzle poses an issue when living cells are incorporated in the ink, and it is still a challenge to integrate cells into macroscopic structures with high precision while maintaining their viability and responsiveness. Furthermore, maintaining the viability of the cells during 3D printing can be difficult due to the fragile nature of the biologics, where common elements in additive manufacturing like UV irradiation and high temperature processing could easily pose irreversible damage to the cells. The highest processing temperature was used by Gonzalez et al, who developed a method to build living materials by 3D printing using agarose at 75° C., but bacteria spores were used to survive the high processing temperature, unlike in the present disclosure where bacteria and vegetative states of the microorganisms are utilized, not spores. Besides 3D printing, microfluidic devices and spinning microfluidics were used to create living cell-laden microcapsules and living fibers highly applicable to smart delivery while electrospinning is especially suitable to the generation of nanofibers and fibrous scaffolds for tissue engineering applications. However, building three-dimensional structures using these methods requires secondary assembly method, leading to further loss of cell viability and functionality.

In our past work, we have demonstrated that biological molecules like protease enzymes and antibiotics can survive thermoplastic molding processing condition at 145° C., 625 MPa and maintained bioactivity when incorporated with silk. In current work, we show, for the first time, that thermoplastic molding as a processing method can be used to engineer living materials-thus, forming dense, plastic-like silk material systems at high temperatures and pressures with living cells encapsulated. This significantly expands the applications that can benefit from living materials, including biosensing, drug delivery platforms, functional implants, renewable surfaces and self-healing interfaces, among others, with easy manufacturing methods, which only requires one-step processing. This invention is also a new strategy to incorporate living microorganisms in silk, where silk is shown to protect the living microorganisms from harsh processing conditions.

Silk has been used to encapsulate/incorporate microorganisms in the past. The use of silk for living materials has been demonstrated by encapsulating microalgae spirulina extract in silk fibroin nanofibers using electrospinning, showing potential as tissue engineering scaffolds that require high hemocompatibility. But only microalgae spirulina extract was used instead of whole cells. Fu et al. have shown that a silk hydrogel can be used to host microalgae as an oxygen generating device and demonstrated that the silk hydrogels with microalgae-embedded in the gel can generate oxygen continuously for 7 days. But these systems were in silk hydrogels, where the processing conditions involve ultrasonication in room temperature and water. These materials are all loose, highly hydrated systems.

As a natural protein-based biopolymer, silk in various material formats has promising features including biocompatibility and biodegradability, along with outstanding mechanical properties. Accordingly, silk has been utilized as a material option for biomaterials and scaffolds in biomedical applications, including drug delivery, tissue engineering and regenerative medicine for decades. Natural silk is a semi-crystalline biopolymer material, consisting of β-sheets nanocrystallites embedded in a less organized, less crystalline continuous phase due to the amphiphilic nature of the protein chemistry. The strong hydrogen-bonding network in the β-sheets nanocrystallites contributes significantly to the stability and excellent mechanical properties of the silks, akin to cellulose, which challenges the ability to thermally process silk-based materials without degradation. In fact, there have only been limited reports of thermal melting and reconstitution of silk materials, which required ultrafast laser heating. Further, historically, starting from Pauling's studies of the fundamental structural features of silk fibroin, the antiparallel β-pleated sheet (antiparallel β-sheet) that form the crystalline phase in silk is significantly stable due to the well aligned N—H—O hydrogen bonds.

Previously known methods of silk protein processing involve solution-based processing; as otherwise, the materials were often found to degrade prior to melting. During the past decades, researchers have made considerable effort in developing techniques to process silk fiber materials with an emphasis on extracting the silk fibroin from fibers and making silk solution. For instance, silk fibers after removing sericin on their exterior surfaces (degummed silk fibers) can be dissolved in aqueous LiBr/CaCl₂) solution with high salt concentrations to generate aqueous silk solution. In addition, degummed silk fibers are soluble in several organic solvents such as formic acid, trifluoroacetic acid, 1,1,1,3,3,3-hexafluoro-2-propanol (HFIP). These methods are aimed towards downstream processing (solvent removal) to generate silk materials, including gels, foams, films, new fiber formats and related materials. Solvent addition and removal, with associated limits of solubility of the protein, lead to new and useful materials, but at a significant cost due to the various processing steps, along with limitations in material properties due to the limits in terms of solubility.

The methods disclosed herein involve the fabrication of amorphous silk nanomaterials (ASN) generated from aqueous silk fibroin solution. ASN may then be treated by hot pressing, leading to fusion and densification of the silk (e.g., into a silk article). The resulting silk bulk material exhibits specific strength higher than that of most natural structural materials and has been shown effective for fabricating silk-based composites. In addition, it is shown that the engineered silk material has thermoforming properties, which allows the materials to be further transformed to desirable shapes under proper conditions. The compositions and methods described herein utilize a thermal and pressure-based, time-efficient and controllable method to transform silk fibroin from a silk fibroin material including substantial amounts of amorphous silk fibroin (for example, in powder form) directly to bulk structural materials. The methods and compositions described herein may allow for the application of more traditional process and molding techniques to silk materials, where this was not previously successfully employed for silk. The described methods involve processes which induce a conformation transition of silk molecules from random coil to β-sheet. In some aspects, provided methods include the processing of natural silk fiber into amorphous silk material (e.g., powder) via degumming, silk fibroin solubilization and freeze drying to prepare the proper premolding materials; feeding the amorphous silk material into a predesigned mold; and inducing the conformation and structure change of silk by applying heat and pressure.

### Compositions

In an aspect of the current disclosure, thermoplastically-molded silk fibroin articles having embedded therein a plurality of microorganisms that are biologically alive and active are provided. The plurality of microorganisms may include bacteria, archaea, fungi, algae, protozoa, viruses, or bacteria (e.g., Escherichia coli, and Gluconacetobacter xylinus). In embodiments, one or more of the plurality of microorganisms produce a biopolymer, such as cellulose.

As used herein, “biologically alive and active” refers to the property of a cell, e.g., a microorganismal cell, to carry out basic biological functions consisting of one or more of DNA replication, RNA transcription, protein translation, and reproduction. Furthermore, “biologically alive and active” includes vegetative forms of microorganisms and may exclude spore or dormant forms of microorganisms, which are especially resistant to heat and pressure.

In one specific case, the plurality of microorganisms of interest include at least one probiotic bacteria. The at least one probiotic bacteria can include a probiotic bacteria having a genus selected from the group consisting of Lactobacillus, Bifidobacterium, Saccharomyces, Streptococcus, Enterococcus, Escherichia, Bacillus, and combinations thereof. The at least one probiotic bacteria can be selected from the group consisting of Escherichia coli, Lactobacillus rhamnosus, Bifidobacterium animalis subs. lactis (Bifidus regularis), and Bifidobacterium longum subs. longum (Bifantis). In some cases, these compositions or articles can further include an additional microorganism of interest that is reactive to the acidic environment of the stomach and that responds to the gastric environment by secreting neutralizing or buffering agents that maintain a lower pH in the vicinity of the composition or article.

In another aspect of the current disclosure, silk living material compositions comprising: a thermoplastically-molded silk fibroin article having embedded therein a plurality of microorganisms of interest that are biologically alive and active, are provided.

The silk articles or compositions disclosed herein may comprise silk, wherein a proportion of the silk fibroin is fused. As used herein “fused” in terms of silk fibroin refers to the generation of silk fibroin that has self-assembled into interlocked nanoglobules made by, for example, treatment with increased temperature and/or pressure.

The disclosed compositions and articles comprising microorganisms which may comprise a heterologous polynucleotide, e.g., a transgene, a plasmid, and the like. The heterologous polynucleotides may comprise a polynucleotide encoding a protein, which may comprise a detectable marker. As used herein, “detectable marker” refers to a marker that is readily identifiable by a particular property of the detectable marker, e.g., electrochemiluminescence, chemiluminescence, fluorescence, radioactivity, nucleotide sequence or amino acid sequence, etc. Exemplary detectable markers include fluorescent markers, e.g., fluorescent proteins, e.g., green fluorescent protein (GFP), red fluorescent protein (RFP) and derivatives of GFP and RFP, and protein purification tags, e.g., Strep tags, HA tags, His tags. Exemplary detectable markers also include enzymes.

The polynucleotide encoding a protein may encode, e.g., a protein for use as a vaccine, e.g., a protein derived from a pathogenic microorganism.

As the disclosed compositions comprise microorganisms that are biologically alive and active, the microorganisms, suitably, express one or more genes. In embodiments, the one or more genes encode a protein, wherein the protein may include a detectable marker, such as a fluorescent marker or an enzyme. Expression of the one or more genes may be under the control of an “exogenous activator”, which increases expression of the one or more genes. Suitable exogenous activators are known in the art, e.g., tetracycline inducible promoter systems (TetON). An exemplary Tet-responsive promoter is described in WO 04/056964A2 (incorporated herein by reference). See, for example, FIG. 1 of WO 04/056964A2. In one construct, a Tet operator sequence (TetOp) is inserted into the promoter region of the vector encoding the disclosed factors. TetOp is preferably inserted upstream of the transcription initiation site, upstream or downstream from the TATA box. In some aspects, the TetOp is immediately adjacent to the TATA box. The expression of the target protein encoding sequence is, thus, under the control of tetracycline (or its derivative doxycycline, or any other tetracycline analogue). Addition of tetracycline or doxycycline (dox) relieves repression of the promoter by a tetracycline repressor that the host cells are also engineered to express. Thus, in such aspects, the inducible factor is tetracycline. Additional exogenous activator systems include cumate-inducible promoters, see, U.S. Pat. No. 10,135,362, rapamycin, abscisic acid and FK506 binding protein 12-based inducible promoter systems are also suitable for use as exogenous activators.

The expression of the one or more genes may be reduced by an exogenous repressor. In the TetOFF system, a different tet transactivator protein is expressed in the tetOFF host cell. The difference is that Tet/Dox, when bind to an activator protein, is now required for transcriptional activation. Thus, such host cells expressing the activator will only activate the transcription of an shRNA encoding sequence from a TetOFF promoter at the presence of Tet or Dox. In embodiments, the one or more genes encode a protein, wherein the protein may include a detectable marker, such as a fluorescent marker or an enzyme. The disclosed articles and compositions comprise a plurality of microorganisms, e.g., bacteria, which may be, for example, Escherichia coli, which is widely available, or Gluconacetobacter xylinus, which may be purchased from the American Type Culture Collection, listed under the designation ATCC 23767. G. xylinus synthesizes cellulose in an appropriate carbohydrate-containing medium, e.g., a medium containing glucose, sucrose, or mannitol. Accordingly, the inventors have demonstrated that the disclosed articles and compositions comprising G. xylinus represent EBLMs that are able to synthesize biopolymers, in this case, cellulose; however, the inventors contemplate that additional or alternative microorganisms producing biopolymers may be used in the disclosed articles and compositions. For example, the inventors contemplate that the disclosed articles and compositions comprise microorganisms producing silk, keratin, and polyhydroxyalkanoates. Such compositions and articles which produce biopolymers may be referred to as self-healing compositions and articles because, if the composition or article is damaged, they are able to produce a polymer to “heal” or repair the damage, see, for example, FIG. 3 which shows cellulose-producing G. xylinus “healing” or repairing a cavity in thermoplastically molded silk compositions. Disclosed herein is a self-healing silk composition including a thermoplastically-molded silk fibroin article having embedded therein a plurality of microorganisms of interest that are biologically alive and active. The plurality of microorganisms of interest produce a polymer compatible with silk, such as cellulose, when the self-healing silk composition is exposed to an expression-compatible culture medium, and optionally, the polymer covers the self-healing silk composition or fills a void in the self-healing silk composition. Generating self-healing silk may involve applying elevated temperature and/or elevated pressure to a mixture of silk fibroin and a plurality of microorganisms.

### Methods

In another aspect of the current disclosure, methods are provided. In some aspects, the methods comprise providing a mixture of silk fibroin and a plurality of microorganisms of interest; and applying at least one of an elevated temperature and an elevated pressure to the silk fibroin-microorganism mixture to generate a thermoplastically-molded silk fibroin article having embedded therein the plurality of microorganisms of interest that are biologically alive and active. Applying may induce fusion between at least a portion of the silk fibroin and structural change in the silk fibroin material.

In one exemplary method, the mixture of silk fibroin and a plurality of microorganisms is lyophilized, generating a powder which is then used for thermoplastic molding. Lyophilizing may include at least one of reduced temperature and reduced pressure.

With methods described herein using heat and pressure-assisted processing of amorphous silk precursor material, different formats of silk materials including plates, rods, screws, and tubes can be prepared with tunable mechanical properties and thermal forming property while retaining the good biocompatibility and degradability features of the materials. In some aspects, methods described herein allow for green and cost-effective methods to transform natural fiber into silk monoliths compared to previous reported methods.

In some cases, the methods described herein can include selecting an elevated temperature and an elevated pressure to produce a desired silk fibroin article of a desired crystallinity and desired material properties and then applying that elevated temperature and elevated pressure to a silk fibroin material having substantially amorphous structure. That is, the methods described herein can predictably select and apply temperatures and pressures to produce articles having desired crystallinity and material properties. This differs from other applications where heat and/or pressure can be applied to silk materials without a pre-determined desired outcome in terms of crystallinity and material properties.

The amount of plasticizer in the silk fibroin material can be adjusted to produce the desired crystallinity and material properties. This is particularly effective in low-temperature aspects, where the amount of plasticizer is selected to produce the desired crystallinity and material properties. In some cases, the plasticizer is water.

In embodiments, the elevated temperature comprises about 100° C. In embodiments, the elevated pressure comprises about 625 MPa of pressure.

The present disclosure also provides methods of use. It should be apparent that these articulated methods of use represent a sub-sample of the full universe of use possibilities for the disclosed articles and compositions.

In one aspect, the present disclosure provides a method of administering a probiotic to a subject in need thereof. The method can include administering a probiotic-containing composition as described herein to a subject in need thereof.

In another aspect, the present disclosure provides a method of treating soil. The method includes: placing into soil the composition or article disclosed herein; and maintaining the soil within a predetermined moisture content range for a predetermined length of time. The placing and maintaining achieves at least two outcomes. First, the placing and maintaining provide to the soil outputs from the one or more organisms. Second, the placing and maintaining provide to the soil a portion of the microorganisms themselves. At least apportion of the thermoplastically-molded silk biodegrades within a predetermined degradation length of time.

### Silk Materials

Any of a variety of silk materials may be used in accordance with various aspects. In some aspects, a silk material may be or comprise silk fibroin (e.g., degummed or substantially sericin free silk fibroin). In some aspects, a silk material may be or comprise silk powder (e.g., comprising a plurality of silk particles).

In some aspects, a silk fibroin material may be or comprise silk particles (e.g., microparticles or nanoparticles). As used herein, the term “particles” includes spheres, rods, shells, prisms, and related structures. While any application-appropriate particle size is contemplated as within the scope of the present disclosure, in some aspects, a silk particle be have a diameter between 1 nm and 1,000 μm (e.g., between 1 nm and 1 μm, between 1 μm and 1,000 μm, etc.). In some aspects, a silk particle may have a diameter of greater than 1,000 μm.

Various methods of producing silk particles (e.g., nanoparticles and microparticles) are known in the art. For example, a milling machine (e.g., a Retsch planetary ball mill) can be used to produce silk powder. Generally, the ball mill consists of either two or four sample cups arranged around a central axis, which is geared such that each cup rotates both centrally and locally. Each ceramic cup is filled with small ceramic spheres. A range of sizes is available; balls with a diameter of 10 millimeters were/are used for the milling operations described in the present disclosure. As the cups spin, the spheres crush material in the cups to a small characteristic size. Both degummed and non-degummed silk can be converted from pulverized material to powder form in the ball mill.

In other aspects, alternative powder formation techniques can be used (e.g., lyophilization or flash freezing and crushing). In other aspects, alternative grates on the pulverizer, with larger holes, can be used. This can generate larger silk particle sizes.

In some aspects, silk particles can be produced using a freeze-drying method as described in U.S. Provisional Application Ser. No. 61/719,146, filed Oct. 26, 2012, the content of which is incorporated herein by reference in its entirety. Specifically, silk foam can be produced by freeze-drying a silk solution. The foam then can be reduced to particles. For example, a silk solution can be cooled to a temperature at which the liquid carrier transforms into a plurality of solid crystals or particles and removing at least some of the plurality of solid crystals or particles to leave a porous silk material (e.g., silk foam). After cooling, liquid carrier can be removed, at least partially, by sublimation, evaporation, and/or lyophilization. In some aspects, the liquid carrier can be removed under reduced pressure. After formation, the silk fibroin foam can be subjected to grinding, cutting, crushing, or any combinations thereof to form silk particles. For example, the silk fibroin foam can be blended in a conventional blender or milled in a ball mill to form silk particles of desired size.

In some aspects, the silk fibroin material comprising substantial amounts of amorphous structure is prepared from silk solution and is composed of nanostructures (as shown in FIG. 1), an may be referred to as nano-sized silk powder (NSP) and be part of materials referred to amorphous silk nanomaterials (ASN). As used herein, these terms are equivalent and may be used interchangeably.

### Silk Fibroin

According to various aspects, any silk fibroin may be used in provided methods. In some aspects, the silk fibroin is selected from the group consisting of spider silk (e.g., from Nephila ciavipes), silkworm silk (e.g., from Bombyx mori), and recombinant silks (e.g., produced/engineered from bacterial cells, yeast cells, mammalian cells, transgenic animals, and/or transgenic plants). In accordance with various embodiments, silk used in provided methods and compositions is degummed silk (i.e. silk fibroin with at least a portion of the native sericin removed). Degummed silk can be prepared by any conventional method known to one skilled in the art. For example, B. mori cocoons are boiled for a period of pre-determined time in an aqueous solution. Generally, longer degumming time generates lower molecular silk fibroin. In some aspects, the silk cocoons are boiled for at least 60 minutes, at least 70 minutes, at least 80 minutes, at least 90 minutes, at least 100 minutes, at least 110 minutes, at least 120 minutes, or longer. Additionally or alternatively, in some aspects, silk cocoons can be heated or boiled at an elevated temperature. For example, in some aspects, silk cocoons can be heated or boiled at about 101.0° C., at about 101.5° C., at about 102.0° C., at about 102.5° C., at about 103.0° C., at about 103.5° C., at about 104.0° C., at about 104.5° C., at about 105.0° C., at about 105.5° C., at about 106.0° C., at about 106.5° C., at about 107.0° C., at about 107.5° C., at about 108.0° C., at about 108.5° C., at about 109.0° C., at about 109.5° C., at about 110.0° C., at about 110.5° C., at about 111.0° C., at about 111.5° C., at about 112.0° C., at about 112.5° C., at about 113.0° C., 113.5° C., at about 114.0° C., at about 114.5° C., at about 115.0° C., at about 115.5° C., at about 116.0° C., at about 116.5° C., at about 117.0° C., at about 117.5° C., at about 118.0° C., at about 118.5° C., at about 119.0° C., at about 119.5° C., at about 120.0° C., or higher. In some aspects, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some aspects, the aqueous solution used in the process of degumming silk cocoons comprises about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some aspects, the extracted silk can be dissolved in about 8 M-12 M LiBr solution. The salt is consequently removed using, for example, dialysis.

In some aspects, the silk fibroin is substantially depleted of its native sericin content (e.g., 5% (w/w) or less residual sericin in the final extracted silk). In some aspects, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some aspects, the silk fibroin is essentially free of its native sericin content. As used herein, the term “essentially free” (or “consisting essentially of”) means that only trace amounts of the substance can be detected, is present in an amount that is below detection, or is absent.

If necessary, a silk solution may be concentrated using, for example, dialysis against a hygroscopic polymer, for example, PEG, a polyethylene oxide, amylose or sericin. In some aspects, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution. In some aspects, a silk fibroin solution can optionally, at a suitable point, be filtered and/or centrifuged. For example, in some aspects, a silk fibroin solution can optionally be filtered and/or centrifuged following the heating or boiling step. In some aspects, a silk fibroin solution can optionally be filtered and/or centrifuged following the dialysis step. In some aspects, a silk fibroin solution can optionally be filtered and/or centrifuged following the step of adjusting concentrations. In some aspects, a silk fibroin solution can optionally be filtered and/or centrifuged following the step of reconstitution. In any of such aspects, the filtration and/or centrifugation step(s) can be carried out to remove insoluble materials. In any of such aspects, the filtration and/or centrifugation step(s) can be carried out to selectively enrich silk fibroin fragments of certain molecular weight(s).

In some aspects, silk fibroin and/or a silk fibroin article, may comprise a protein structure that substantially includes β-turn and/or β-strand regions. Without wishing to be bound by a theory, the silk β sheet content can impact gel function and in vivo longevity of the composition. It is to be understood that composition including non-β sheet content (e.g., e-gels) can also be utilized. In some aspects, silk fibroin has a protein structure including, e.g., about 5% β-turn and β-strand regions, about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, silk fibroin has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% β-turn and β-strand regions, or at least 95% β-turn and β-strand regions. In yet other aspects of these embodiments, silk fibroin has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some aspects, silk β sheet content, from less than 10% to ~55% can be used in the silk fibroin compositions disclosed herein.

In some aspects, silk fibroin, or a silk fibroin article, has a protein structure that is substantially-free of α-helix and/or random coil regions. In aspects of these embodiments, the silk fibroin has a protein structure including, e.g., about 5% α-helix and/or random coil regions, about 10% α-helix and/or random coil regions, about 15% α-helix and/or random coil regions, about 20% α-helix and/or random coil regions, about 25% α-helix and/or random coil regions, about 30% α-helix and/or random coil regions, about 35% α-helix and/or random coil regions, about 40% α-helix and/or random coil regions, about 45% α-helix and/or random coil regions, or about 50% α-helix and/or random coil regions. In other aspects of these embodiments, the silk fibroin has a protein structure including, e.g., at most 5% α-helix and/or random coil regions, at most 10% α-helix and/or random coil regions, at most 15% α-helix and/or random coil regions, at most 20% α-helix and/or random coil regions, at most 25% α-helix and/or random coil regions, at most 30% α-helix and/or random coil regions, at most 35% α-helix and/or random coil regions, at most 40% α-helix and/or random coil regions, at most 45% α-helix and/or random coil regions, or at most 50% α-helix and/or random coil regions. In yet other aspects of these embodiments, the silk fibroin has a protein structure including, e.g., about 5% to about 10% α-helix and/or random coil regions, about 5% to about 15% α-helix and/or random coil regions, about 5% to about 20% α-helix and/or random coil regions, about 5% to about 25% α-helix and/or random coil regions, about 5% to about 30% α-helix and/or random coil regions, about 5% to about 40% α-helix and/or random coil regions, about 5% to about 50% α-helix and/or random coil regions, about 10% to about 20% α-helix and/or random coil regions, about 10% to about 30% α-helix and/or random coil regions, about 15% to about 25% α-helix and/or random coil regions, about 15% to about 30% α-helix and/or random coil regions, or about 15% to about 35% α-helix and/or random coil regions.

### Elevated Temperatures

As discussed herein, provided methods and compositions include the exposure to elevated temperature(s). As used herein, the term “elevated temperatures” refers to temperatures higher than standard room temperature (i.e., greater than 25° C.). In some aspects, provided methods or compositions include exposure to a single elevated temperature. In some aspects, provided methods or compositions include exposure to at least two elevated temperatures (e.g., 3, 4, 5, 6, 7, 8, 9, 10 or more). In some aspects where a method of composition includes two or more elevated temperatures, at least two of those elevated temperatures are different from one another.

In some aspects, an elevated temperature may be between 25° C. and 200° C. By way of specific exemplary ranges, in some aspects, an elevated temperature may be between 25° C. and 150° C., between 25° C. and 100° C., between 25° C. and 95° C., between 25° C. and 50° C., between 50° C. and 200° C., between 50° C. and 150° C., between 50° C. and 100° C., between 25° C. and 100° C. . . . between 125° C. and 200° C., or any other range between 125° C. and 175° C.

In some aspects, an elevated temperature may be at least 25° C. By way of additional example, in some aspects, an elevated temperature may be at least 26° C., 27° C., 28° C., 29° C., 30° C., 35° C., 40° C., 45° C., 50° C., 55° C., 60° C., 65° C., 70° C., 75° C., 80° C., 85° C., 90° C., 95° C. or 100° C. In some aspects, enhanced crystallization of silk fibroin material is observed at temperatures at or above 95° C.

In some aspects, an elevated temperature may be at most 125° C. By way of additional example, in some aspects, an elevated temperature may be at most 126° C., 127° C., 128° C., 129° C., 130° C., 135° C., 140° C., 145° C., 150° C., 155° C., 160° C., 165° C., 170° C., 175° C., 180° C., 185° C., 190° C., or 195° C.

Application of elevated temperature(s) to a provided composition or in a provided method may occur in any application-appropriate manner. By way of non-limiting example, in some aspects, application of elevated temperature(s) may be via heat pressing, via a heating device such as an oven, heating stage, exposed flame or other mechanism.

Application of elevated temperature(s) may occur at or over any of a variety of time periods. For example, in some aspects, application of elevated temperature(s) occurs substantially instantly (e.g., by placement over a flame or in an oven). In some aspects, application of elevated temperature(s) occurs over a period of seconds, minutes, or hours. In some aspects, application of elevated temperature(s) occurs over a period of time between 1 second and 1 hour.

### Elevated Pressure

As discussed herein, provided methods and compositions include the exposure to elevated pressure(s). As used herein, the term “elevated pressures” refers to pressures higher than standard atmospheric pressure (i.e., 1.013 bar). In some aspects, provided methods or compositions include exposure to a single elevated pressure. In some aspects, provided methods or compositions include exposure to at least two elevated pressures (e.g., 3, 4, 5, 6, 7, 8, 9, 10 or more). In some aspects where a method of composition includes two or more elevated pressures, at least two of those elevated pressures are different from one another.

Any application-appropriate method(s) may be used to cause elevated pressure as applied to provided compositions or in provided methods. By way of non-limiting example, in some aspects, elevated pressure may include use of a vacuum, a press (e.g. heat press), and combinations thereof.

In some aspects, application of elevated pressure may be or include uniaxial compression. In some aspects, application of elevated pressure may be or include multi-axial compression (e.g., biaxial compression).

While any application-appropriate level of elevated pressure may be used, in some aspects, an elevated pressure between 1 MPa and 1 GPa is used. By way of specific exemplary ranges, in some aspects, an elevated pressure may be between 10 MPa and 1 GPa, between 50 MPa and 1 GPa, between 100 MPa and 1 GPa, between 200 MPa and 1 GPa, between 300 MPa and 1GP, between 400 MPa and 1 GPa or between 500 MPa and 1 GPa. In some aspects, an elevated pressure may be or comprise at least 1 MPa (e.g., at least 2 MPa, 3 MPa, 4 MPa, 5 MPa, 6 MPa, 7 MPa, 8 MPa, 9 MPa, 10 MPa, 20 MPa, 30 MPa, 40 MPa, 50 MPa, 60 MPa, 70 MPa, 80 MPa, 90 MPa, 100 MPa 150 MPa, 200 MPa, 250 MPa, 300 MPa, 350 MPa, 400 MPa, 450 MPa, 500 MPa, 550 MPa, 600 MPa, 650 MPa, 700 MPa, or 750 MPa).

### Silk Articles

The provided methods and compositions allow for the production of engineered biologic living materials (EBLMs), also referred to herein as “silk articles”. In some aspects, the provided EBLMs exhibit a substantially homogenous structure. As used herein, “substantially homogenous structure” means that silk fibroin molecules are distributed and/or configured in a consistent way throughout substantially all of a portion of or the entirety of an article (e.g., such as wherein the dispersed plurality of microorganisms of interest are detected in a cross section of the composition). Further, in some aspects, EBLMs may exhibit significant amounts of silk fibroin in a semi-crystalline structure. In some aspects, production of EBLMs or silk articles according to provided methods includes a transition on the structure of silk fibroin from a substantially amorphous state to a semi-crystalline state, for example, as observed via X-ray diffraction.

In some aspects, a silk article may exhibit significant amounts of β-sheet structure. For example, in some aspects, a silk article may exhibit at least 10 wt % more (e.g., at least 20 wt %, 30 wt %, 40 wt %) β-sheet structures as compared to the starting silk fibroin material. In some aspects, a silk article may exhibit at least 50 wt % more (e.g., at least 60 wt %, 70 wt %, 80 wt %, 90 wt %, 95 wt %) β-sheet structures as compared to the starting silk fibroin material.

In some aspects, crystallinity of silk articles may be controlled by the application of temperature and pressure. For example, in some aspects, when amorphous silk is processed at temperatures ranging from about 25° C.-125° C., the silk article may contain about 10-15% β-sheet structures. In some aspects when amorphous silk is processed at temperatures ranging from about 125° C.-175° C., the silk article may contain for example, about 20-35% β-sheet structures or for example, over 40% β-sheet structures.

In some aspects, provided methods and compositions allow for the production of silk articles which that are homogenous, where the silk amorphous powders are packed together via the bonding between neighboring raw silk powders, for example, at processing temperatures of about 25° C.-95° C. In some aspects, provided methods and compositions allow for the production of silk articles which that are homogenous, where the silk molecules of amorphous powders gain more mobility as they are heated above the glass transition temperature and self-assemble into interlocked nanoglobules, for example, at processing temperatures of about 125° C.-175° C.

In some aspects, provided methods and compositions allow for the production of silk articles (e.g., thin films) that undergo thermal softening and are bendable and moldable into a desired shape. In some aspects, provided methods and compositions allow for the production of silk articles that are machinable.

Provided methods and compositions allow for the production of complex silk articles (e.g., silk screws that can resist torsion forces relevant to in vivo use). By way of non-limiting example, in some aspects provided methods and compositions may be used to produce silk articles such as films, fibers, meshes, needles, tubes, plates, screws, rods, and any combination thereof, further comprising living cells.

In some aspects, a silk article may be amenable to one or more types of patterning. In some aspects, patterning may be or comprise macropatterning. In some aspects, patterning may be or comprise micropatterning (i.e., patterning with micro scale features). In some aspects, patterning may be or comprise nanopatterning (i.e., patterning with nano scale features). In some aspects, patterning may be or comprise: etching, lithography-based patterning, carving, cutting, and any combination thereof.

In some aspects, a silk article may be subjected to one or more types of processing (e.g., 2, 3, 4, 5, 6, 7, 8, 9, 10 or more). While any application-appropriate form of processing is contemplated as within the scope of the present disclosure, in some aspects, processing may be or comprise machining, rolling, drilling, milling, sanding, punching die cutting, extruding, chemical etching, coating, molding, turning, thread rolling, and any combination thereof.

### Exemplary Properties or Characteristics of Silk Articles

In some aspects, provided compositions (e.g., silk articles) may be substantially transparent. In some aspects, provided compositions (e.g., silk articles) may be semi-transparent. In some aspects, provided compositions (e.g., silk articles) may be substantially non-transparent. As used herein, the term “transparent” refers to the propensity of an object to transmit light (with or without scattering of said light). In some aspects, a composition/article is said to be substantially transparent if it transmits ≥80% of light it is exposed to in the visible range (400 nm-800 nm). In some aspects, a composition/article is said to be semi-transparent if it transmits between 50%-80% of light it is exposed to in the visible range (400 nm-800 nm). In some aspects, a composition/article is said to be substantially non-transparent if it transmits≤50% of light it is exposed to in the visible range (400 nm-800 nm).

In some aspects, provided compositions may be biocompatible and/or biodegradable. In some aspects, provided compositions may exhibit particular degradation profile(s). By way of specific example, in some aspects, a provided composition may degrade at least 50% by weight after about 96 hours of exposure to an aqueous environment at 37° C. In some aspects, a provided composition may not degrade more than 10% after months of exposure to an in vivo environment or condition.

In some aspects, provided compositions may exhibit one or more desirable properties including, but not limited to: electrical conductivity, enhanced machinability, and/or enhanced thermoformability.

In some cases, provided compositions or articles can have a bulk density that is tailored to be higher than hydrogel compositions. In some cases, the compositions or articles disclosed herein can have a bulk density of between 1.0 kg/dm³ and 3.0 kg/dm³. In some cases, the articles or compositions can have a bulk density of 1.4 kg/dm³.

The provided compositions or articles have a water content that is significantly lower than the water content of hydrogels. Without wishing to be bound by any particular theory, it is believed that the higher levels of hydration in hydrogels may make it a more receptive environment for living microorganisms than a thermoplastically-moldable silk material that has a lower water content. It was not clear before conducting the experiments whether microorganisms could be maintained as biologically alive and active in such a low water content environment. The inventors unexpectedly discovered that the present disclosure provides the capacity to produce thermoplastically-molded silk fibroin articles or compositions where the water content by weight is less than 50%, less than 40%, less than 30%, or less than 20%.

The resulting thermoplastically-molded silk has a structure that is characterized by granular structures surrounding individual microorganisms.

### Additives

In some aspects, provided methods and compositions include one or more additives (e.g., 2, 3, 4, 5, 6, 7, 8, 9, 10 or more). In some aspects, at least one additive may be mixed with or otherwise associated with a silk fibroin material prior to an applying step (e.g. exposure to one or more of elevated temperature and elevated pressure). In some aspects, at least one additive may be mixed with or otherwise associated with a silk fibroin material substantially at the same time as an applying step). In some aspects, at least one additive may be mixed with or otherwise associated with a silk fibroin material subsequently to an applying step. As used herein, “additive” comprises a compound and does not refer to living cells.

Provided methods and compositions are amenable to the addition of any of a variety of additives. By way of non-limiting example, in some aspects an additive may be or comprise a small molecule, an organic macromolecule, an inorganic macromolecule, an electrically conductive material, an inorganic material, a hydrophobic material, a hydrophilic material, a nanomaterial, and any combination thereof.

The processing of the silk-based materials, including pure silk materials and silk-based composite materials, can be modified with addition of one or more additives. In some aspects, a function of an additive may be to tune the processing conditions and the properties of the products. In some aspects, additives may be selected from water; glycerol; saccharides; biological macromolecules, e.g. peptide, proteins; antibodies and antigen binding fragments; nucleic acids; immunogens; antigens; enzyme; synthetic polymers, e. g. poly(ethylene) glycol, poly-lactic acid, poly(lactic-co-glycolic acid) to name but a few specific examples, though any application-appropriate additive is specifically contemplated as within the scope of the present disclosure.

In some aspects, for example some aspects contemplated for in vivo use, provided compositions may comprise one or more proteases. In some aspects, an organic macromolecule is or comprises at least one protease. In some aspects, a protease is or comprises one or more of Proteinase XIV, Proteinase K, a-chymotrypsin, collagenase, matrix metalloproteinase-1 (MMP-1), and MMP-2. In some aspects, a protease may be useful in tailoring the degradation profile of a particular provided composition (e.g., in an in vivo environment).

In some aspects, an electrically conductive material may be or comprise an organic conductive material and/or an inorganic conductive material (e.g., a metal). In some aspects, an electrically conductive material may be or comprise at least one of a conductive polymer, graphene, silver, gold, aluminum, copper, platinum, steel, brass, bronze, and iron oxide.

Any application-appropriate amount of one or more additives may be useful according to various aspects. By way of non-limiting example, in some aspects, an additive may be present in a provided composition in an amount between 0.001 wt % and 95 wt %. In some aspects, one or more additives may be mixed with a silk fibroin material in an amount ranging between 0.001 wt % and 95 wt % of the silk fibroin material.

### EXAMPLES

### Example 1—E. coli Cells are Viable after Thermoplastic Molding in Silk Plate

### Methods

### Regenerated Bacteria Containing Silk Fibroin Preparation.

Bombyx mori cocoons were cut into small pieces and boiled in an aqueous 0.02M Na2CO3 (Sigma-Aldrich) solution for 30 min, followed by rinsing in distilled water to remove the Na2CO3 and sericin. The degummed silk was allowed to dry at room temperature overnight. The dried silk was dissolved in 9.3M LiBr (Sigma-Aldrich) solution at 60° C. for 3-4 h. The solution was subsequently dialyzed for 3d in distilled water using Slide-a-Lyzer dialysis cassettes (MWCO 3,500). The water was changed five times during the dialysis (1 h, 4 h, 8 h, 24 h, 48 h). After dialysis, the solution was centrifuged for 20 min at 9,780 g twice to remove insoluble impurities. The concentration of the final silk solution was determined by measuring a volume of solution and the final dried weight (~6w/v %).

For the E. coli containing materials, E. coli was inoculated into lysogeny both (LB, 10 g/L Tryptone, 5 g/L yeast extract, and 10 g/L NaCl) media supplemented with 100 μg/mL ampicillin and grown overnight. The optical density at 600 nm (OD₆₀₀) was recorded to be 3.4, and 10 mL of bacteria were pelleted for 10 min at 4° C. and 3,000×g. In subsequent experiments the volume of E. coli pelleted was normalized to its OD₆₀₀ and the initial experiment to keep the number of bacteria consistent for each trial

$$( {V}_{E . coli} = \frac{10 mL \times 34}{{OD}_{600}} ) .$$

The silk solution was diluted to 1% silk (w/v) and mixed with bacteria pellets and frozen using liquid nitrogen. The frozen silk solution was lyophilized at −80° C. and 0.006 bar until complete sublimation.

### Hot Pressing of Bacteria Contained Silk Materials

Lyophilized silk material containing bacteria (lyophilized as above) was packed into predesigned molds, followed by hot pressing at 625 MPa and 100° C. After hot pressing, the samples were cooled down to room temperature and used for incubation.

### Viability Test

E. coli strain NEB10β (New England Biolabs, Ipswich, MA) harboring ampicillin resistant plasmid pET25b (+) (Novagen, Darmstadt, Germany) was used for viability testing. The thermoplastic molded plates with and without bacteria, and thermoplastic molded bacteria as controls were immersed into Luria-Bertani (LB) broth (1 ml) containing 100 μg/mL ampicillin. 100 μL supernatant for each sample was collected after 16 hours of inoculation. The supernatant was diluted subsequently to 10⁻¹, 10⁻², 10⁻³, 10⁻⁴, and 10⁻⁵ times. Then 5 μL each of the series of dilutions was deposited on ampicillin contained LB agar plates and inoculated for 12 hours to monitor bacterial colony formation. The supernatant for green fluorescent protein (GFP) expressing E. coli (E. coli strain Nissle 1917 (Mutaflor®, ECN)) containing silk plates inoculation was collected for microscopic fluorescent imaging (BZ-X700 Fluorescence Microscope (Keyence Corp., Itasca, IL)) at excitation wavelength of 488 nm.

### Results

FIG. 1A shows the process of making silk living materials using thermoplastic molding. In this process, the viability of bacteria could be compromised. First, dissolving bacteria pellets into silk aqueous solution (1 wt %) with no incorporation of media needed for bacteria could potentially lead to loss of viability. After dissolution, the bacterial silk mixture is put into −80° C. freeze overnight. The freezing process can cause viability loss as well. The frozen silk bacteria mixture is then transferred for lyophilization until completely dried for the thermoplastic process. The resulting sponge is then thermoplastic molded into dense silk materials with desired shape for further applications. These parts can be machined as well if needed for further refinement of shape and features.

The impact of the process to the bacteria viability is shown in FIG. 1B. We have prepared four sets of samples with the same mass: silk sponge pressed at room temperature and ambient pressure without thermoplastic molding, silk plate pressed at 100° C. at 625 MPa pressure as thermoplastic molding conditions, silk sponge containing ampicillin resistant E. coli pressed at room temperature at ambient pressure, and silk sponge containing ampicillin resistant E. coli thermoplastic molded at 100° C., 625 MPa. We found that E. coli survived the freeze-drying conditions after dissolution in silk aqueous solution as bacterial colonies are observed with dilutions as low as 10⁻⁵. The thermoplastic molded silk plates containing E. coli also show bacterial colonies, although with a lower viability according to the decreased colonies at the series of dilutions compared with the plates without thermoplastic molding. No colonies are observed for samples containing no bacteria and samples containing only E. coli but processed following the same procedure of thermoplastic molding (FIG. 1C).

To confirm the survival of E. coli after thermoplastic molding, we incorporated GFP expressing E. coli following the same protocol as above. After overnight inoculation, we collected the supernatant of the media and monitored the fluorescent intensity using a fluorescence microscope. The fluorescent intensity correlated with the number of bacteria expressing GFP in the media. As shown in FIG. 1D, silk plates with and without thermoplastic molding show fluorescence intensity after overnight inoculation, while wild type E. coli and silk only controls show no fluorescence, demonstrating that bacteria survive the thermoplastic molding process and have the potential to be processed in this way as living materials, as they still express the GFP after thermoplastic processing.

### Example 2—E. Coli are Responsive to Stimulation after Thermoplastic Molding in Silk Plate

### Methods

mRFP-E. coli were obtained by transformation of commercially available BLR(DE3) (Novagen) using the pBAD-mRFP1vector (Addgene plasmid #91765) provided as a bacterial agar stab. Bacteria from the agar stab was streaked into an agar plate supplemented with ampicillin (100 μL/mL) and let to grow overnight at 37° C. Isolated colonies were selected and grown in Luria Broth (ThermoFisher, Cat. N. J75854.A1) supplemented with 100 μL/mL of ampicillin overnight at 37° C. 250 rpm in an orbital shaker. Bacteria was collected and plasmid was purified (GeneJET PCR Purification Kit, ThermoFisher Scientific, Waltham, Massachusetts, USA). 50 μL of BLR(DE3) we transformed with 80 ng of pure pNCS-mRFP plasmid and plated into agar plates supplemented with ampicillin. Isolated colonies were grown in TB autoinduction media (BocaScientific Cat. N. GCM19.0500) supplemented with 8 mL/L of glycerol for 8 hours at 37° C. and 250 rpm in an orbital shaker. Bacteria were than analyzed by SDS-PAGE in order to screen for the best producing colony which was subsequently used for the storage in glycerol stock. For viability tests, mRFP-E. coli were grown in LB media supplemented with ampicillin for 16 hours at 37° C. to achieve a high optical density (OD=8-10).

The optical density at 600 nm (OD₆₀₀) of mRFP-E. coli was adjusted to be 3.4, and 10 mL of bacteria were pelleted for 10 min at 4° C. and 3,000×g. In subsequent experiments the volume of E. coli pelleted was normalized to its OD₆₀₀ and the initial experiment to keep the number of bacteria consistent for each trial.

mRFP-E. coli was incorporated into silk sponges following the above protocol. The thermoplastic plates were immersed in LB broth containing 100 μg/mL ampicillin. The plates were imaged before and after inoculation for 16 hours and 4 days using a fluorescence microscope (BZ-X700 Fluorescence Microscope (Keyence Corp., Itasca, IL)) at excitation wavelength of 532 nm. At day 5, the inoculated plates were transferred into an induction media (LB media containing 100 μg/mL of ampicillin and 20 μg/mL lactose). Lactose is used as an additional carbon source for the mRFP-E. coli and to mildly upregulate the T7 promotor-based expression of the mRFP. Fluorescent images were taken after further inoculation at day 6. All pictures are taken with the same imaging settings including excitation exposure time (1/20 s). Scanning electron microscopy (SEM) was used to image the E. coli on the surface and in the cross-section after inoculation for 6 days.

### Results

FIG. 2 shows the red fluorescence intensity significantly increased after inoculation for 4 days. With added lactose, the intensity shows further enhancement. This result demonstrated that the bacteria that are trapped in the silk plates are not only viable, but also are able to proliferate and function (expressing mRFP). From 16 h to day 4, the fluorescence intensity enhancement is a result of bacterial cell proliferation as more cell growth results in higher fluorescent intensity. The significant enhancement of fluorescence after adding lactose is a result of cell expressing red fluorescent proteins as lactose is known to induce the expression. We used SEM to examine if there are E. coli present after inoculation. As shown in FIG. 2E and FIG. 2F, the E. coli are present both on surface and in cross-section.

### Example 3—Self-Healing of Silk Plates Comprising Polymer Generating Microorganisms

### Methods

Gluconacetobacter xylinus (ATCC 23767) was rehydrated with approximately 0.5 mL of mannitol broth. The solution was then be transferred to a 5-6 mL tube containing mannitol broth and incubated at 26° C. for 2-4 days. The desired amount of G. xylinus was centrifuged to obtain the bacteria pellet to be used. G. xylinus containing silk sponge was prepared following the above procedure. A cavity was created in the middle of the sponge as shown in FIG. 3B for control plates and thermoplastic molded plates. The plates were inoculated in mannitol broth for 10 days to allow cellulose growth on the plate. The inoculated plates were ETO autoclaved and lyophilized. Scanning electron microscopy images were taken to show the cellulose formation in the cavity to fill the defect.

### Results

FIG. 3B shows that the silk plates containing cellulose bacteria with cavities created in the middle were healing (filling in) through cellulose growth in the cavity. The cellulose growth was observed for both plates with thermoplastic molding and without thermoplastic molding. SEM images confirm the fibers formed in the cavity is cellulose fibers (FIG. 3C). FIG. 3D shows the digital images of the control experiment, where thermoplastic molded silk plate with a cavity was immersed in the same media for the same period and no cavity healing was observed. This experiment demonstrates that the cellulose bacteria-containing silk living material is functional where G. xylinus are viable, functional, and possess self-healing capability due to the bacterial production of cellulose in the void or on surfaces.

### Example 4—Viability Test of E. coli Encapsulated Silk from Thermoplastic Molding Comparing with Other Biopolymers

### Methods

E. coli strain NEB10β (New England Biolabs, Ipswich, MA) harboring ampicillin resistant plasmid pET25b(+) (Novagen, Darmstadt, Germany) was used for viability testing. The biopolymers silk fibroin, sodium alginate (Sigma Aldrich), gelatin (Sigma Aldrich), soy (Bulk supplements.com), chitosan (Sigma Aldrich), cellulose (Sigma Aldrich), and zein (Sigma Aldrich) were mixed with same concentration of bacteria aqueous solution (the quantity of bacteria was controlled as same as FIG. 1). The mixture was freeze dried. Lyophilized biopolymer material containing bacteria (lyophilized as above) was packed into predesigned molds, followed by hot pressing at 625 MPa and 100° C. After hot pressing, the samples were cooled down to room temperature and used for incubation. The viability test was conducted after overnight incubation with 5 times series of dilutions on LB-ampicillin agar plates.

### Results

As shown in FIG. 4A, after thermoplastic molding at 625 MPa and 100° C., only silk fibroin encapsulated E. coli maintained viability. The other biopolymers did not provide the same stabilization outcome as the silk. The digital images of thermoplastic molded plates (FIG. 4B-C) show that thermoplastic molding of silk fibroin result in transparent plates that remained intact after incubation, suitable for living materials applications. Meanwhile, besides failing to stabilize bacteria in thermoplastic molding conditions, alginate, cellulose, chitosan, and zein do not fuse into transparent plates after molding, and the alginate and gelatin failed to remain integrity by dissolving in the media.

### Example 5—E. Coli Cells are Viable after Thermoplastic Molding in a Gastrointestinal Environment and Soil Environment

### Methods

### Preparation of Silk Living Materials

Non-pathogenic E. coli Nissle 1917 (EcN) strain was kindly donated by Prof. Christopher A. Voigt MIT. The pDawn plasmid was a gift from Andreas Moeglich (Addgene plasmid #43796; http://n2t.net/addgene: 43796; RRID: Addgene_43796). A red fluorescence protein (RFP) gene was amplified via polymerase chain reaction (PCR) using primer sequences GTAGTACATATGGCCTCCTCCGAGGAC (SEQ ID 1) and GATGATCTCGAGTTAGGCGCCGGTGGAGTG (SEQ ID 2). This RFP gene and the pDawn plasmid were digested with NdeI and XhoI restriction enzymes (Thermo Fisher, USA) and ligated using standard and commercially established molecular biology techniques to prepare the pDawn-RFP reporter plasmid. Transformed colonies were selected on Luria-Bertani (LB) agar (Fisher Scientific, USA) containing 50 μg/mL kanamycin (Kan). The EcN with pDawn-RFP strain (REcN) was initially inoculated in 5 mL of LB Kan medium and was cultivated at 37° C. and 250 rpm for 18 h. Next, the REcN cell culture was used to inoculate a new larger flask containing desired volumes of LB Kan medium, and these were grown up to an OD600 of 1 under the same culture conditions. The cell pellets were collected by centrifuging 30 mL of REcN cell culture with OD600 of 1 at 3,500 rpm and 4° C. for 10 min, and the supernatant was discarded. Bacteria were collected and washed with PBS before mixing with pre-prepared silk solutions. Finally, 30 mL of 1 wt % silk solution was uniformly mixed with the centrifuged REcN cell pellets, frozen, and lyophilized at −80° C. and 0.006 bar.

Rhizobacteria (Rhizobium tropici CIAT 899 Martinez-Romero et al., ATCC 49672) were cultivated in Rhizobium X medium according to the ATCC handling information. CIAT. 899 (CIAT. 899) was cultivated to the OD600 reach 1 at 30° C. and 250 rpm. Like the REcN silk composites preparation described above, 30 mL of 1 wt % silk solution was mixed with the cell pellets obtained by centrifuging 30 mL of CIAT. 899 cell culture with an OD600 value of 1 at 9,000 rpm at 4° C. for 20 min. Subsequently, the CIAT. 899 and silk mixture was freeze-dried at −80° C. and 0.006 bar.

### Thermal Molding of Bacteria Contained Silk Materials

TS/REcN were prepared by direct thermal molding silk/REcN powders at 632 MPa and 60° C. for 15 min. The WS/REcN or WS/CIAT. 899 were prepared by molding water mist plasticized silk/REcN or silk/CIAT. 899 powder for 15 min prior thermal molding at 632 MPa and 60° C. for 15 min.

### Viability Test

Simulated gastric fluid (0.2% sodium chloride in 0.7% hydrochloric acid) with pH of 1.0-1.4 (SGF, Ricca Chemical, USA) supplemented with 3.2 mg/mL of pepsin from porcine gastric mucosa (Sigma-Aldrich, USA) and simulated intestinal fluid (pancreatin, potassium dihydrogen phosphate and sodium hydroxide) with pH of 6.7-6.9 (SIF, Ricca Chemical, USA). Naked REcN (5×10⁸ CFU) and lyophilized WS/REcN and TS/REcN with equal amounts of 5×10⁸ CFU were subjected to the 1 mL of SGF and further cultured at 250 rpm in a 37° C. incubator for 2 h, respectively. In addition, to better mimic the real gastrointestinal tract, the same amount of naked REcN, WS/RECN, and TS/REcN as previous described were incubated in SGF for 2 h. Then, the naked REcN collected by centrifugation (5,000 rpm, 5 min), WS/REcN, and TS/REcN were further incubated in 1 mL of SIF for 2 h. After different treatments, naked REcN were centrifuged and washed twice with PBS and the resuspended naked REcN in LB/Kan medium were plated on solid LB in sequential dilutions of 10 with an overnight incubation at 37° C. for bacterial colonies counting. At the same time, treated WS/REcN and TS/REcN were incubated in a culture medium for the 1 week of REcN release assessments, the treated WS/REcN and TS/REcN were transferred to the new culture medium every 24 h, supernatants collected at each 24 h for the bacterial counting. For proliferation activity assessments, the optical density value at 600 nm (OD600) of the naked REcN and collected REN after the different treatments were diluted to 0.05, and the OD600 values of each sample were recorded at 30 30-minute intervals by the microplate reader (Varioskan™ LUX multimode, ThermoFisher Scientific, USA) for the growth curves plot. For fluorescence measurements, excitation and emission wavelengths were set to 554 nm and 591 nm to monitor the RFP density changes of the REcN by a microplate reader. In addition, naked REcN and treated REcN collected after different treatments were stained with the Live & Dead Bacterial Staining Kit (Invitrogen, USA) and detected by a fluorescence microscope (BZ-X700, Keyence, USA).

### Protease Activity

WS/60° C. and WS/CIAT.899 were incubated in a 30 mL of Rhizobium X medium at room temperature as previously described. At 7-day intervals, 100 μL supernatant was used to measure protease activity using the Protease Activity Assay Kit (Abcam, USA).

### Degradation Measurements

WS/60° C. and WS/CIAT.899 were loaded in nylon mesh bags with a pore size of 100 μm under standard environmental conditions and then placed in soil with a moisture content of 25% for a degradation period of 90 days.

The degraded samples were cleaned and dried before weighing. Five samples per group were used for soil degradation study, and five samples were used for each time point. WS/60° C. degradation was performed by incubating the WS/60° C. samples (n=3) in 1 U/mL protease XIV at 37° C. (Sigma-Aldrich, USA). For each sample, 2 mL of protease XIV incubation solution was used and changed every 2 days. After rinsing with deionized water and drying, the remaining mass of the sample after incubation was recorded. Degradation under soil and protease XIV conditions is assessed monthly gravimetrically by initial sample mass and corresponding mass after degradation.

### Finite Element Analysis

To understand the stress and temperature distribution behaviors during the experimental thermal molding process, FEA analysis with built-in modules of Structural Mechanics and MEMS were performed using commercial software (COMSOL Multiphysics 6.1.). To simplify the computational complexity, the optimized analysis model consisted of microbe spheres with diameter of 1 μm randomly placed inside the silk cylinder (3 mm height, 2 mm diameter), with a volume ratio of total microbe spheres to cylinder is 1:9. Both the spheres and cylinder were molded as the linear elastic materials. Parameters of the experimentally obtained Young's modulus were adopted for simulation. Fixed constraint, boundary load and roller constraint were set on the outside of the geometry in solid mechanics based on the thermal molding process; roller constraint was assigned to reinforce the deformation in the X, Y direction to zero. In the heat transfer in solids field, the processing temperature was set to 60° C. The solid mechanics field and heat transfer in solids field were coupled by the thermal expansion field. In order to accurately analysis the stress and temperature changes within the cylinder and sphere during the thermal molding process, a free tetrahedral mesh was introduced in the mesh with an average element size close to one for refined mesh division. Additionally, a time-dependent solver is set up for the simulation model with the backward differentiation formulation to ensure an accurate control of the time-steps, and the relative tolerance of the model is set to 0.001.

### Results

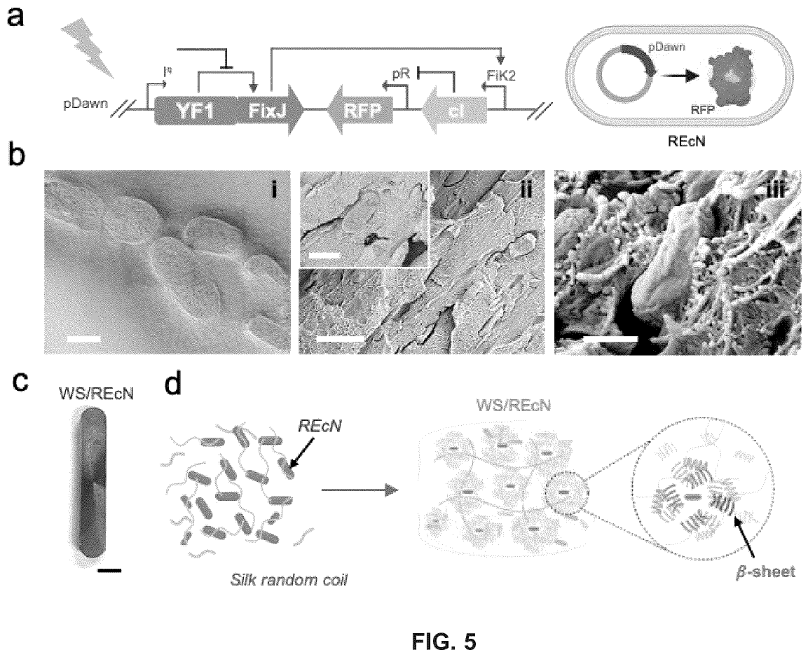

As living microorganisms, probiotics face harsh conditions in the gastrointestinal tract, including low pH, digestive enzymes, and bile salts. Consequently, designing a protective delivery system enables the preservation of the viability and function of probiotics against degradation and deactivation in these harsh conditions. Plasticizer-assisted thermal molding of silk with high β-sheet crystallinity is disclosed herein as a probiotic delivery system, with the gut probiotic EcN as a model bacterium for the evaluation of oral administration probiotic efficacy. For better imaging, the optogenetic protein expression plasmid, pDawn with a red fluorescent protein (RFP) gene, to construct the pDawn-RFP reporter plasmid (FIG. 5a). SEM images of the lyophilized silk/REcN powder revealed rod-shaped REcNs encapsulated with a layer of silk coating before molding (FIG. 5b i). In addition, WS/REcN obtained through the plasticizer-assisted thermal molding of silk/REcN powder were observed, with numerous REcNs randomly embedded in the granular silk (FIG. 5b ii). Individual RECN with intact rod-like structures were surrounded by the granular silk matrix, as evidenced by sharper high-magnification SEM images (FIG. 5b iii). Meanwhile, the morphology of silk changed from sheet to granular structures, suggesting the high crystallinity transition of silk supports the protective shell surrounding the bacteria (FIG. 5c).

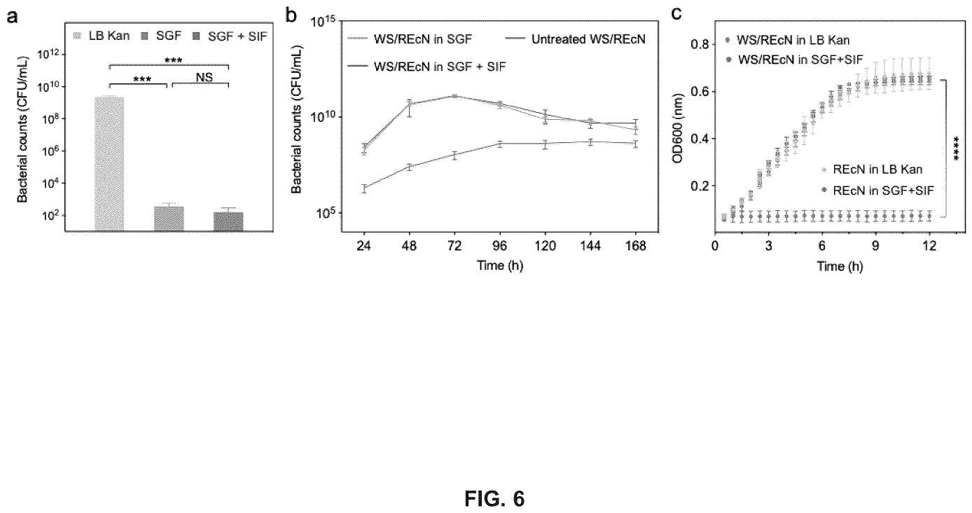

Furthermore, in vitro assessment of REcN was conducted with acidic simulated gastric fluid supplemented with pepsin (SGF, pH 1.2) which is considered the primary challenge to probiotic oral administration. Additionally, to mimic the gastrointestinal tract environment, after culture in SGF for 2 h, the REcN were collected and incubated in simulated intestinal fluid containing pancreatin (SIF, pH 6.8) for another 2 h incubation (termed as SGF+SIF). REcN preserved in Luria-Bertani containing kanamycin (LB Kan) medium were used as the control group. The fluorescence density and survival rates of the REN significantly decreased after both the SGF or SGF+SIF incubations compared to the control group, suggesting the plasmid damage and lethality to REcN due to the acidic gastric environment (FIG. 6a).

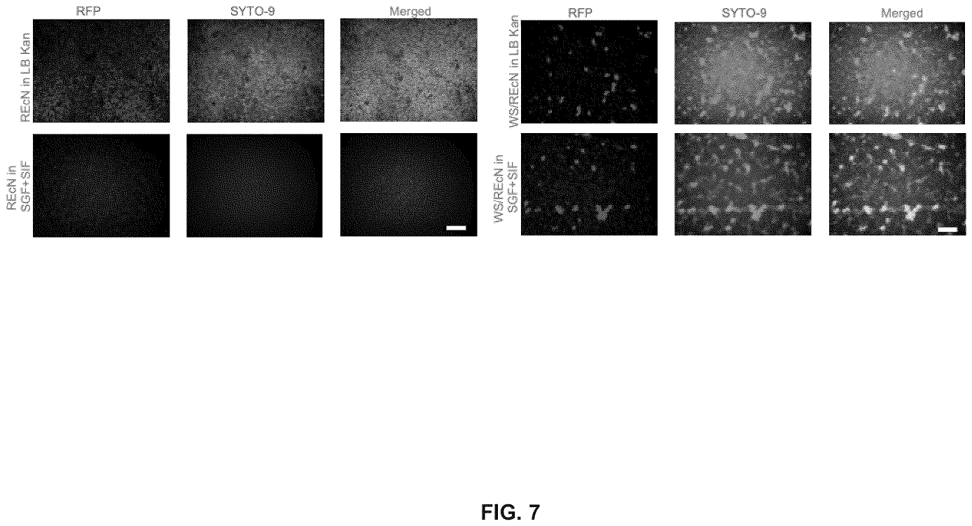

The proliferation of RECN from WS/REcN was further evaluated after one week of incubation in LB Kan medium following SGF or SGF+SIF treatment. As shown in FIG. 6b, REcN was released and proliferated from WS/REcN within 24 h after simulated acidic gastric treatment. It is noteworthy that the number of REcN was higher in the SGF or SGF+SIF-treated groups during the evaluation period compared to untreated WS/REcN, which may be related to partial silk degradation caused by acids and digestive enzymes in SGF or SGF+SIF. Moreover, the amount of live RECN released in the SGF and SGF+SIF treated groups remained comparable to the untreated WS/REN group over the extended period. These results suggested that the compact silk structure with more ordered crystallinity prevented the direct exposure of live microorganisms to environmental stressors and thus maintained the viability of live probiotic bacteria, as compared to looser structures with a lower degree of crystallinity. As shown in the OD600 growth curves, the growth of RECN released from SGF+SIF-treated WS/REcN after 7 days of culture was similar to that in LB Kan medium or released from the untreated WS/REN, while significantly higher than the REcN group treated with SGF and SIF, in which almost no living cells were detected (FIG. 6c). Furthermore, fluorescence microscopy images visually confirmed that the majority of REcN in LB Kan medium or collected from untreated WS/REcN or SGF+SIF-treated WS/REcN were active, which contrasted with naked REcN treated with SGF+SIF. These observations are consistent with the microbial activities of each group described previously (FIG. 7).

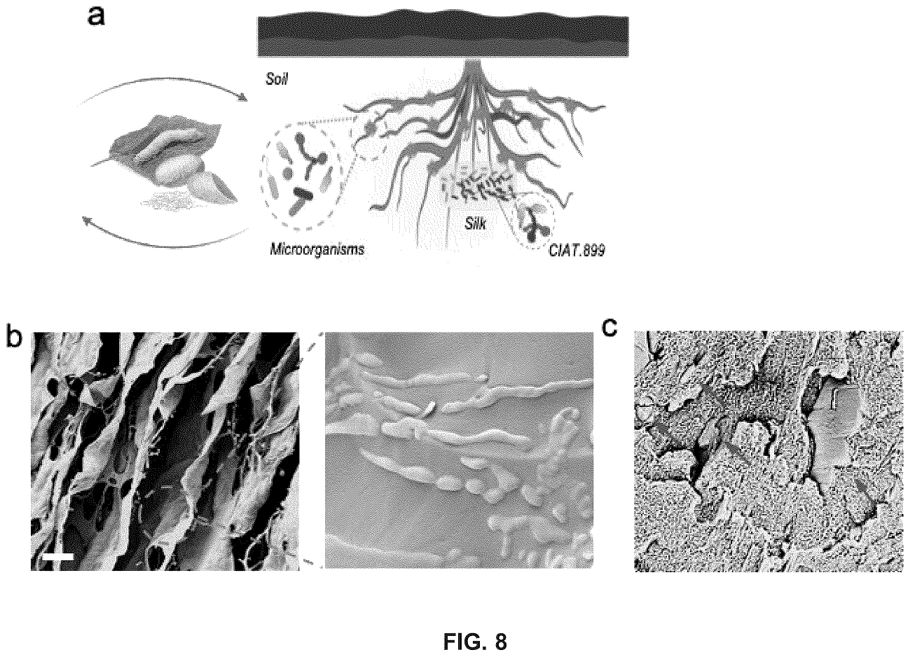

In embodiments, efficient biodegradability and sustainability are criteria for the development of new bioplastic materials, providing potential perspectives to address the problem of plastic waste disposal. To further explore low-cost and efficient methods for silk degradation, and simultaneously to leverage silk as a bioresource, rhizobacterial strains were embedded in silk as a living system (FIG. 8a). Previous studies have confirmed that active bacteria secreted proteases and induced local pH acidification when exposed to moist soil. As disclosed herein, Rhizobium tropici CIAT. 899 (CIAT. 899) as a model bacterium was incorporated into silk bioplastics to construct a hybrid silk/CIAT. 899 living system, followed by an evaluation of degradation in soil. SEM images indicated the presence of CIAT. 899 encapsulated by the silk sheets before the molding process (FIG. 8b). The structure of WS/CIAT. 899 was observed (FIG. 8c), and the sheet-like structures of silk were transformed into granular morphologies post plasticizer-assisted thermal molding processing, allowing the CIAT. 899 to be densely armored in these ordered conformations of the silk matrix.

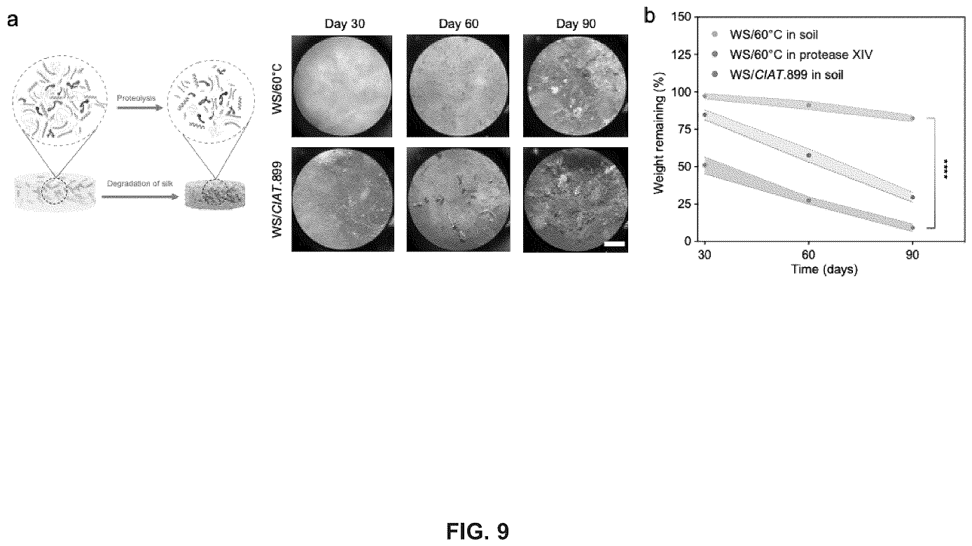

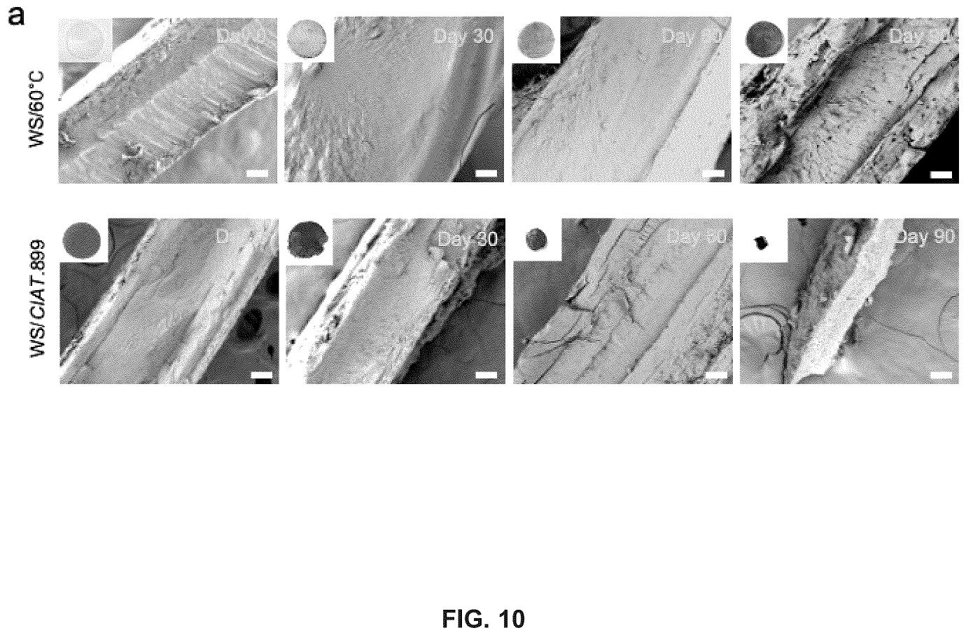

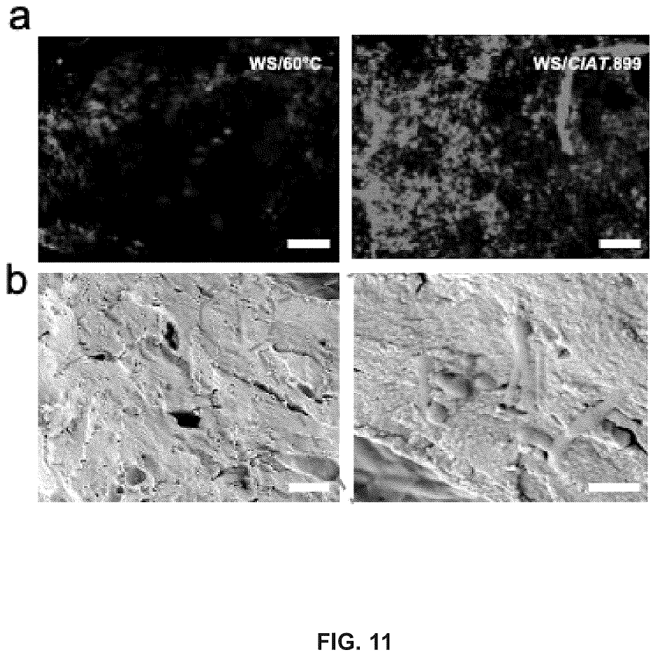

The proteases secreted by active CIAT. 899 induced hydrolysis of silk proteins. The evolution of WS/CIAT. 899 degradation was investigated for 90 days in soil. The soil degradation behavior of the hybrid WS/CIAT. 899 and abiotic WS/60° C. as the control were observed at different stages by optical microscopy (FIG. 9a). This observation suggested that the degradation of the WS/CIAT. 899 living system in soil was more efficient (FIG. 9b). The gradually decrease in thickness and the looser structure of WS/CIAT. 899, as evident from the SEM images in FIG. 10, with 90 days demonstrating the morphological transformation of WS/CIAT. 899 from a stiff plastic to a sludge-like brown residue compost, while the relatively intact WS/60° C. appeared only slightly eroded on the surface. Furthermore, the examination of live cells through fluorescence microscopy imaging stained with green fluorescence, as well as SEM imaging, supported the persistence and colonization of CIAT. 899 encased in the silk bioplastics after a 90-day soil cultivation period (FIGS. 11a and 11b). These results indicated the development of a sustained silk living materials platform with enhanced microbial retention.

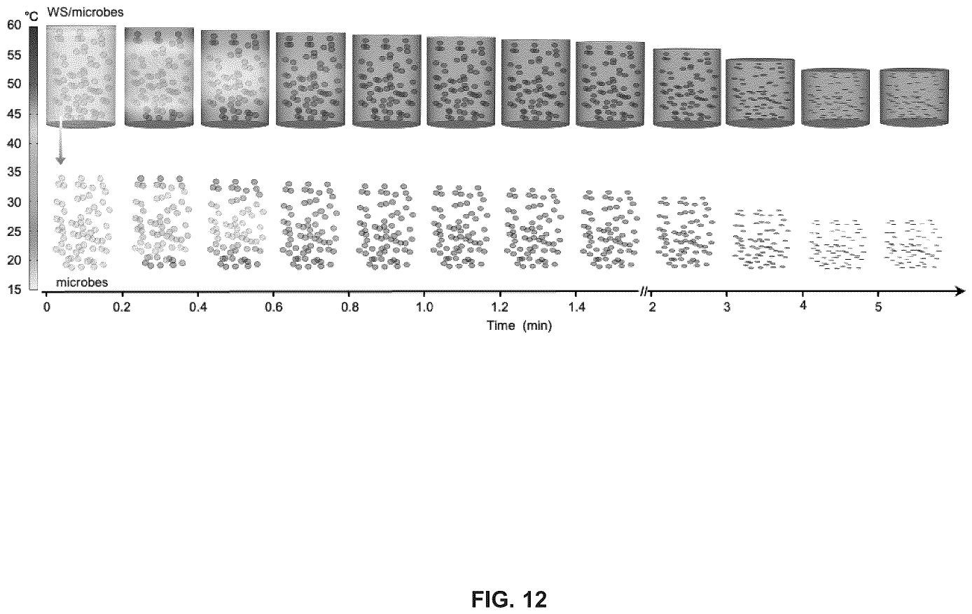

A simplified plasticizer-assisted thermal molding system was developed for silk and microbial composites (WS/microbes), and finite element analysis (FEA) was employed to gain insight into the distribution of applied external temperature and molding stress during the process. The deformations throughout the molding process were characterized by the von Mises stress function over time, revealing a gradual increase in stress that reached peak after 4 minutes of the total molding event. FIG. 12 illustrates the evolution of the WS/microbe model influenced by heating and molding, exhibiting good agreement with experimental observations. These modeling results offer guidance for the construction of silk-based living material systems incorporating diverse biotic components.

Equivalents and Scope. The recitation of a listing of elements in any definition of a variable herein includes definitions of that variable as any single element or combinations (or sub-combinations) of listed elements. The recitation of an embodiment herein includes that embodiment as any single embodiment or in combination with any other embodiments or portions thereof. Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

Aspects of the disclosure include the following statements:

1. A thermoplastically-molded silk fibroin article having embedded therein a plurality of microorganisms of interest that are biologically alive and active.

2. A silk living material composition comprising:
a thermoplastically-molded silk fibroin article having embedded therein a plurality of microorganisms of interest that are biologically alive and active.

3 An article comprising the composition of clause 1 or 2.

4. The article or composition of any one of the preceding clauses, wherein the composition is substantially homogenous.

5. The article or composition of any one of the preceding clauses, wherein a proportion of the silk fibroin is fused.

6. The article or composition of any one of the preceding clauses, wherein the plurality of microorganisms of interest are dispersed within the composition.

7. The article or composition of clause 6, wherein the dispersed plurality of microorganisms of interest are detected in a cross section of the composition.

8. The article or composition of any one of the preceding clauses, wherein at least one of the plurality of microorganisms of interest comprises a heterologous polynucleotide.

9. The article or composition of clause 8, wherein the heterologous polynucleotide comprises a polynucleotide encoding a protein.

10. The article or composition of clause 9, wherein the protein comprises a detectable marker.

11. The article or composition of clause 10, wherein the detectable marker comprises a fluorescent marker or an enzyme.

12. The article or composition of any one of the preceding clauses, wherein at least one of the plurality of microorganisms of interest expresses one or more genes.

13. The article or composition of clause 12, wherein expression of the one or more genes is induced by an exogenous activator.

14. The article or composition of clause 12, wherein expression of the one or more genes is reduced by an exogenous repressor.

15. The article or composition of clause 13 or 14, wherein the one or more genes encode a protein.

16. The article or composition of clause 15, wherein the protein comprises a detectable marker.

17. The article or composition of clause 16, wherein the detectable marker is a fluorescent marker or an enzyme.

18. The article or composition of any one of the preceding clauses, wherein the plurality of microorganisms of interest comprises one or more microorganisms selected from the group consisting of bacteria, archaea, fungi, algae, protozoa, and viruses.

19. The article or composition of clause 18, wherein the one or more microorganisms comprise bacteria.

20. The article or composition of clause 19, the bacteria include at least one probiotic bacteria.

21. The article or composition of clause 20, wherein the at least one probiotic bacteria includes a probiotic bacteria having a genus selected from the group consisting of Lactobacillus, Bifidobacterium, Saccharomyces, Streptococcus, Enterococcus, Escherichia, Bacillus, and combinations thereof.

22. The article or composition of clause 21, wherein the at least one probiotic bacteria includes a probiotic bacteria selected from the group consisting of Escherichia coli, Lactobacillus rhamnosus, Bifidobacterium animalis subs. lactis (Bifidus regularis), and Bifidobacterium longum subs. longum (Bifantis).

23. The article or composition of any one of clauses 20 to 22, wherein the one or more microorganisms further comprises an acid-reactive microorganism that metabolizes within an acidic environment to produce reactants that lower the pH in the vicinity of the article or composition.

24. The article or composition of clause 19, wherein the bacteria are selected from Escherichia coli, and Gluconacetobacter xylinus.

25. The article or composition of clause 24, wherein the bacteria are Gluconacetobacter xylinus.

26. The article or composition of any one of clauses 1-21, wherein the plurality of microorganisms of interest produces a biopolymer.

27. The article or composition of clause 22, wherein the biopolymer is cellulose.

28. The article or composition of any one of the preceding clauses, wherein the thermoplastically-molded silk has a bulk density of between 1.0 kg/dm³ and 3.0 kg/dm³.

29. The article or composition of any one of the preceding clauses, wherein the thermoplastically-molded silk has a total water content by weight of less than 50%, less than 40%, less than 30%, or less than 20%.

30. The article or composition of any one of the preceding clauses, wherein the thermoplastically-molded silk is characterized by a granular structure independently surrounding individual microorganisms of at least a portion of the one or more microorganisms.

31. The article or composition of clause 30, wherein the thermoplastically-molded silk has a degree of crystallinity of at least 50%, at least 60%, at least 70%, or at least 75%.

32. A self-healing silk composition comprising:
a thermoplastically-molded silk fibroin article having embedded therein a plurality of microorganisms of interest that are biologically alive and active.

33. The self-healing silk composition of clause 24, wherein the plurality of microorganisms of interest produces a polymer compatible with silk when the self-healing silk composition is exposed to an expression-compatible culture medium and, optionally, wherein the polymer covers the self-healing silk composition or fills a void in the self-healing silk composition.

34. An article comprising the composition of clause 32 or 33.

35. The article or self-healing silk composition of any one of clauses 32 to 34, wherein the polymer is cellulose.

36. The article or self-healing silk composition of any one of clauses 32 to 35, wherein the composition is substantially homogenous.

37. The article or self-healing silk composition of any one of clauses 32 to 36, wherein a proportion of the silk fibroin is fused.

38. The article or self-healing silk composition of any one of clauses 32 to 37, wherein the plurality of microorganisms of interest are dispersed within the composition.

39. The article or self-healing silk composition of clause 38, wherein the dispersed plurality of microorganisms of interest are detected in a cross section of the composition.

40. The article or self-healing composition of any one of clauses 32 to 39, wherein at least one of the plurality of microorganisms of interest comprises a heterologous polynucleotide.

41. The article or self-healing silk composition of clause 40, wherein the heterologous polynucleotide comprises a polynucleotide encoding a protein.

42. The article or self-healing silk composition of clause 41, wherein the protein comprises a detectable marker.

43. The article or self-healing silk composition of clause 42, wherein the detectable marker comprises a fluorescent marker or an enzyme.

44. The article or self-healing silk composition of any one of clauses 32 to 43, wherein at least one of the plurality of microorganisms of interest expresses one or more genes.

45. The article or self-healing silk composition of clause 44, wherein expression of the one or more genes is induced by an exogenous activator.

46. The article or self-healing silk composition of clause 44, wherein expression of the one or more genes is reduced by an exogenous repressor.

47. The article or self-healing silk composition of clause 45 or 46, wherein the one or more genes encode a protein.

48. The article or self-healing silk composition of clause 47, wherein the protein comprises a detectable marker.

49. The article or self-healing silk composition of clause 48, wherein the detectable marker is a fluorescent marker or an enzyme.

50. The article or self-healing silk composition of any one of clauses 32 to 49, wherein the plurality of microorganisms of interest comprises one or more microorganisms selected from the group consisting of bacteria, archaea, fungi, algae, protozoa, and viruses.

51. The article or self-healing silk composition of clause 50, wherein the one or more microorganisms comprise bacteria.

52. The article or self-healing silk composition of clause 51, wherein the bacteria are selected from Escherichia coli, and Gluconacetobacter xylinus.

53. The article or self-healing silk composition of clause 52, wherein the bacteria are Gluconacetobacter xylinus.

54. The article or self-healing silk composition of any one of clauses 32 to 53, wherein the plurality of microorganisms of interest produces a biopolymer.

55. The article or self-healing silk composition of clause 54, wherein the biopolymer is cellulose.

56. A method comprising:
providing a mixture of silk fibroin and a plurality of microorganisms of interest; and
applying at least one of an elevated temperature and an elevated pressure to the silk fibroin-microorganism mixture to generate a thermoplastically-molded silk fibroin article having embedded therein the plurality of microorganisms of interest that are biologically alive and active.

57. A method of generating a silk living material comprising:
providing a mixture of silk fibroin and a plurality of microorganisms of interest; and
applying at least one of an elevated temperature and an elevated pressure to the mixture of silk fibroin and a plurality of microorganisms to generate a silk living material having embedded therein the plurality of microorganisms of interest that are biologically alive and active.

58. The method of any one of clauses 56 to 57, wherein the mixture of silk fibroin and a plurality of microorganisms of interest has a substantially amorphous structure.

59. The method of any one of clauses 56 to 58, wherein the mixture of silk fibroin and a plurality of microorganisms of interest is lyophilized.

60. The method of any one of clauses 56 to 58, wherein the method further comprises after providing a mixture of silk fibroin and a plurality of microorganisms of interest:
lyophilizing the mixture of silk fibroin and the plurality of microorganisms of interest.

61. The method of clause 60, wherein the lyophilizing comprises at least one of reduced temperature and reduced pressure.

62. The method of clause 61, wherein the lyophilizing comprising both reduced temperature and reduced pressure.

63. The method of any one of clauses 56 to 62, wherein the elevated temperature is 100° C. 64. The method of any one of clauses 56 to 63, wherein the elevated pressure is 625 Mpa of pressure.

65. The method of any one of clauses 56 to 64, wherein the applying step comprises application of both elevated temperature and elevated pressure to the silk fibroin material.

66. The method of any one of clauses 56 to 65, wherein the applying step induces fusion between at least a portion of the silk fibroin and structural change in the silk fibroin material.

67. The method of any one of clauses 56 to 66, wherein the applying step is or comprises heat pressing.

68. The method of clause 67, wherein the heat pressing occurs at a pressure that is at least 1 MPa.

69. The method of clause 67 or 68, wherein the heat pressing occurs at a temperature that is between 25° C. and 200° C., wherein the heat pressing optionally occurs at a temperature that is 100° C.

70. The method of any one of clauses 56 to 69, wherein at least one of the plurality of microorganisms of interest comprises a heterologous polynucleotide.

71. The method of clause 70, wherein the heterologous polynucleotide comprises a polynucleotide encoding a protein.

72. The method of clause 71, wherein the protein comprises a detectable marker.

73. The method of clause 72, wherein the detectable marker comprises a fluorescent marker or an enzyme.

74. The method of any one of clauses 56 to 73, wherein at least one of the plurality of microorganisms of interest expresses one or more genes after the applying step.

75. The method of clause 74, wherein expression of the one or more genes is induced by an exogenous activator.

76. The method of clause 75, wherein expression of the one or more genes is reduced by an exogenous repressor.

77. The method of clause 75 or 76, wherein the one or more genes encode a protein.

78. The method of clause 77, wherein the protein comprises a detectable marker.

79. The method of clause 78, wherein the detectable marker is a fluorescent marker or an enzyme.

80. The method of any one of clauses 56 to 79, wherein the plurality of microorganisms of interest comprises one or more microorganisms selected from the group consisting of bacteria, archaea, fungi, protists, and viruses.

81. The method of clause 80, wherein the one or more microorganisms includes one or more bacteria.

82. The method of clause 81, wherein the one or more microorganisms comprise bacteria.

83. The method of clause 82, the bacteria include at least one probiotic bacteria.

84. The method of clause 83, wherein the at least one probiotic bacteria includes a probiotic bacteria having a genus selected from the group consisting of Lactobacillus, Bifidobacterium, Saccharomyces, Streptococcus, Enterococcus, Escherichia, Bacillus, and combinations thereof.

85. The method of clause 84, wherein the at least one probiotic bacteria includes a probiotic bacteria selected from the group consisting of Escherichia coli, Lactobacillus rhamnosus, Bifidobacterium animalis subs. lactis (Bifidus regularis), and Bifidobacterium longum subs. longum (Bifantis).

86. The method of clause 81, wherein the bacteria are selected from Escherichia coli, and Gluconacetobacter xylinus.

87. A method of generating self-healing silk comprising:
providing a mixture of silk fibroin and a plurality of microorganisms of interest; and
applying at least one of an elevated temperature and an elevated pressure to the mixture of silk fibroin and a plurality of microorganisms to generate self-healing silk.

88. The method of clause 87, wherein the plurality of microorganisms are biologically alive and active.

89. The method of any one of clauses 87 to 88, wherein the mixture of silk fibroin and the plurality of microorganisms of interest has a substantially amorphous structure.

90. The method of any one of clauses 87 to 89, wherein the mixture of silk fibroin and the plurality of microorganisms of interest is lyophilized.

91. The method of any one of clauses 87 to 89, wherein the method further comprises after providing a mixture of silk fibroin and the plurality of microorganisms of interest:
lyophilizing the mixture of silk fibroin and the plurality of microorganisms of interest.

92. The method of clause 91, wherein the lyophilizing comprises at least one of reduced temperature and reduced pressure.

93. The method of clause 92, wherein the lyophilizing comprises both reduced temperature and reduced pressure.

94. The method of any one of clauses 87 to 93, wherein the elevated temperature is 100° C. 95. The method of any one of clauses 87 to 94, wherein the elevated pressure is 625 Mpa of pressure.

96. The method of any one of clauses 87 to 95, wherein the applying step comprises application of both elevated temperature and elevated pressure to the silk fibroin material.

97. The method of any one of clauses 87 to 96, wherein the applying step induces fusion between at least a portion of the silk fibroin and structural change in the silk fibroin material.

98. The method of any one of clauses 87 to 97, wherein the applying step is or comprises heat pressing.

99. The method of clause 98, wherein the heat pressing occurs at a pressure that is at least 1 Mpa. 100. The method of clause 98 or 99, wherein the heat pressing occurs at a temperature that is between 25° C. and 200° C., wherein the heat pressing optionally occurs at a temperature that is 100° C.

101. The method of any one of clauses 87 to 100, wherein at least one of the plurality of microorganisms of interest comprises a heterologous polynucleotide.

102. The method of clause 101, wherein the heterologous polynucleotide comprises a polynucleotide encoding a protein.

103. The method of clause 102, wherein the protein comprises a detectable marker.

104. The method of clause 103, wherein the detectable marker comprises a fluorescent marker or an enzyme.

105. The method of any one of clauses 87 to 104, wherein at least one of the plurality of microorganisms of interest expresses one or more genes after the applying step.

106. The method of clause 105, wherein expression of the one or more genes is induced by an exogenous activator.

107. The method of clause 106, wherein expression of the one or more genes is reduced by an exogenous repressor.

108. The method of clause 106 or 107, wherein the one or more genes encode a protein.

109. The method of clause 108, wherein the protein comprises a detectable marker.

110. The method of clause 109, wherein the detectable marker is a fluorescent marker or an enzyme.

111. The method of any one of clauses 87 to 110, wherein the plurality of microorganisms of interest comprises one or more microorganisms selected from the group consisting of bacteria, archaea, fungi, protists, and viruses.

112. The method of clause 111, wherein the one or more microorganisms includes one or more bacteria.

113. The method of clause 112, wherein the bacteria are selected from Escherichia coli, and Gluconacetobacter xylinus.

114. A composition generated by the method of any one of clauses 87 to 113.

115. A method of treating soil, the method comprising:
placing into the soil the composition or article of or made by the method of any the preceding clauses; and
maintaining the soil within a predetermined moisture content range for a predetermined maintaining length of time,
   wherein the placing and maintaining provide to the soil: (i) outputs made from the one or more microorganisms; and/or (ii) at least a portion of the one or more microorganisms,
   wherein at least a portion of the thermoplastically-molded silk biodegrades within the predetermined degradation length of time.

116. The method of clause 115, wherein the one or more microorganisms further includes an enzymatic degradation microorganism that metabolically secretes an enzyme capable of accelerating biodegradation of the thermoplastically-molded silk.

117. A method of delivering a probiotic, the method comprising orally administering to a subject the composition or article of or made by the method of any one of clauses 20 to 23 or 83 to 85.

1. A thermoplastically-molded silk fibroin article having embedded therein a plurality of microorganisms of interest that are biologically alive and active.

2.-4. (canceled)

5. The article of claim 1, wherein a proportion of the silk fibroin in the thermoplastically-molded silk fibroin article is fused.

6. The article of claim 1, wherein the plurality of microorganisms of interest are dispersed within the thermoplastically-molded silk fibroin article.

7. The article of claim 6, wherein the dispersed plurality of microorganisms of interest are detected in a cross section of the thermoplastically-molded silk fibroin article, optionally wherein at least one of the plurality of microorganisms of interest comprises a heterologous polynucleotide, optionally wherein the heterologous polynucleotide comprises a polynucleotide encoding a protein, optionally wherein the protein comprises a detectable marker, optionally wherein the detectable marker comprises a fluorescent marker or an enzyme.

8.-11. (canceled)

12. The article of claim 1, wherein at least one of the plurality of microorganisms of interest expresses one or more genes.

13. The article of claim 12, wherein expression of the one or more genes is induced by an exogenous activator and/or reduced by an exogenous repressor, optionally wherein the one or more genes encode a protein.

14.-17. (canceled)

18. The article of claim 1, wherein the plurality of microorganisms of interest comprises one or more microorganisms selected from the group consisting of bacteria, archaea, fungi, algae, protozoa, and viruses.

19. The article of claim 18, wherein the one or more microorganisms comprise bacteria.

20. The article of claim 18, wherein the one or more microorganisms comprise at least one probiotic bacteria.

21. The article of claim 20, wherein the at least one probiotic bacteria includes a probiotic bacteria having a genus selected from the group consisting of Lactobacillus, Bifidobacterium, Saccharomyces, Streptococcus, Enterococcus, Escherichia, Bacillus, and combinations thereof.

22. The article of claim 21, wherein the at least one probiotic bacteria includes a probiotic bacteria selected from the group consisting of Escherichia coli, Lactobacillus rhamnosus, Bifidobacterium animalis subs. lactis (Bifidus regularis), and Bifidobacterium longum subs. longum (Bifantis).

23. The article of claim 18, wherein the one or more microorganisms further comprises an acid-reactive microorganism that metabolizes within an acidic environment to produce reactants that lower the pH in a vicinity of the article.

24. The article of claim 19, wherein the bacteria are selected from Escherichia coli, and Gluconacetobacter xylinus.

25. The article of claim 24, wherein the bacteria are Gluconacetobacter xylinus.

26. The article of claim 1, wherein the plurality of microorganisms of interest produces a biopolymer.

27. (canceled)

28. The article of claim 1, wherein the thermoplastically-molded silk fibroin article has a bulk density of between 1.0 kg/dm³ and 3.0 kg/dm³ and/or a total water content by weight of less than 50%, less than 40%, less than 30%, or less than 20%.

29. (canceled)

30. The article of claim 1, wherein the thermoplastically-molded silk fibroin article is characterized by a granular structure independently surrounding individual microorganisms of at least a portion of the plurality of microorganisms of interest.

31. The article of claim 30, wherein the thermoplastically-molded silk fibroin article has a degree of crystallinity of at least 50%, at least 60%, at least 70%, or at least 75%.

32. A method comprising:
providing a mixture of silk fibroin and a plurality of microorganisms of interest; and
applying at least one of an elevated temperature and an elevated pressure to the silk fibroin-microorganism mixture to generate a thermoplastically-molded silk fibroin article having embedded therein the plurality of microorganisms of interest that are biologically alive and active.

33.-43. (canceled)

44. A method of treating soil, the method comprising:
placing into the soil a thermoplastically-molded silk fibroin article having embedded therein a plurality of microorganisms of interest that are biologically alive and active; and
maintaining the soil within a predetermined moisture content range for a predetermined maintaining length of time,
wherein the placing and maintaining provide to the soil: (i) outputs made from the plurality of microorganisms; and/or (ii) at least a portion of the plurality of microorganisms,
wherein at least a portion of the thermoplastically-molded silk fibroin article biodegrades within a predetermined degradation length of time.

45.-46. (canceled)


SEQUENCE LISTING (2 sequences)

SEQ ID NO: 1 - DNA, 27 bp, synthetic construct
  source 1..27: mol_type = other DNA
gtagtacatatggcctcctccgaggac

SEQ ID NO: 2 - DNA, 30 bp, synthetic construct
  source 1..30: mol_type = other DNA
gatgatctcgagttaggcgccggtggagtg